(19) United States
(12) Patent Application Publication
(10) Pub. No.: US 2007/0275961 A1
(43) Pub. Date: Nov. 29, 2007

(54) Triazolo ' 1, 5-A ! Pyrimidines and Their Use in Medicine

(75) Inventors: Justin Fairfield Bower, Cambridge (GB); Andrew Cansfield, Cambridge (GB); Allan Jordan, Cambridge (GB); Martin Parratt, Cambridge (GB); Lee Walmsley, Cambridge (GB); Douglas Williamson, Cambridge (GB)

Correspondence Address: BANNER & WITCOFF, LTD., 1100 13th STREET, N.W., SUITE 1200, WASHINGTON, DC, 20005-4051, US

(73) Assignee: VERNALIS (CAMBRIDGE) LIMITED., Abington, GBN (GB)

(21) Appl. No.: 10/558,694

(22) PCT Filed: Jun. 2, 2004
(86) PCT No.: PCT/GB04/02342

§ 371 Date: Mar. 23, 2007

(30) Foreign Application Priority Data
Jun. 4, 2003 (GB) 0312822.0
Jan. 16, 2004 (GB) 0400998.1

(51) Int. Cl. A61K 31/519; A61K 31/496; A61K 31/5377; A61P 17/06; A61P 35/00; A61P 9/00; C07D 413/14; C07D 487/04

(52) U.S. Cl. 514/233.2; 514/252.16; 514/259.31; 544/118; 544/263

(57) ABSTRACT

Compounds of formula (IA) or (AB) are kinase inhibitors, especially of CDK2, and/or PDK1 and/or CHK1: wherein Ring A is an optionally substituted aryl, heteroaryl, carbocyclic or heterocyclic radical, Alk represents an optionally substituted clivaient C₁-C₆ alkylene radical; n is 0 or 1; Q represents a radical of formula -(Alk¹)p-(X),(Alk²)ₛ-Z wherein in any compatible combination Z is hydrogen or an optionally substituted carbocyclic or heterocyclic ring, p, r and s are 0 or 1, and Alk¹, Alk₂, X, are as described in the specification, and R₁ represents a radical 3 4 3 (CYC)ₖ-(Alk³)ₐ-(Y)_{b}-(Alk⁴)_{d}-B wherein k, a, b and d are 0 or 1, and Cyc, Alk³, Alk⁴ and B are as described in the specification.

This invention relates to the use of a class of substituted amino triazolo[1,5-a]pyrimidines in relation to diseases which are mediated by excessive or inappropriate kinase activity, for example CDK2 and/or PDK1 and/or Chk1 activity, such as cancers.

### BACKGROUND TO THE INVENTION

CDK2

Uncontrolled cell proliferation is a hallmark of cancer. Tumor cells typically have damage to genes which play a part in regulation of the cell division cycle. Cyclin-dependent kinases (CDKs) play critical roles in regulating the transitions between different phases of the cell cycle. The serine/threonine kinase CDK2 is essential for normal cell cycling, and plays a key role in disorders arising form aberrant cell cycling. Inhibitors of CDK2 are therefore useful for the treatment of various types of cancer and other conditions related to abnormal cell proliferation. Flavopyridol (M. D. Losiewicz et al, Biochem. Biophys. Res. Commun., 1994, 201, 589-595), which is currently in clinical trials, displays modest selectivity for inhibition of CDKs over other kinases but inhibits CDK1, CDK2, and CDK4 with equal potency. A purine based derivative, roscovitine (CYC-202) (W. F. de Azevedo et al, Eur. J. Biochem., 1997, 243, 518-526), similarly displays selectivity for CDKs over other kinases and is also in clinical trials.

PDK1

For a normal cell to acquire the phenotype of a malignant tumour cell, several barriers must be overcome. One of the most important is the ability to evade programmed cell death (apoptosis). Mutations downregulating various aspects of the cell-death machinery are therefore a hallmark of cancer. The PI-3 kinase-AKT pathway transmits survival signals from growth factor receptors to downstream effectors. In a substantial number of tumour cells, this pathway is inappropriately activated by either amplification of the PI-3 kinase or AKT genes, or loss of expression of the PTEN tumour suppressor. Activation of this pathway enables cancer cells to survive under conditions where normal cells would die, enabling the continued expansion of the tumour. The 3′-phosphoinositide-dependent protein kinase-1 (PDK1) is an essential component of the PI-3 kinase-AKT pathway. In the presence of PIP3, the second messenger generated by PI-3 kinase, PDK1 phosphorylates AKT on threonine 308, a modification essential for AKT activation. PDK1 also phosphorylates the corresponding threonine residues of certain other pro-survival kinases including SGK and p70 S6 kinase (B. Vanhaesebroeck and D. R. Alessi, Biochem. J., 2000, 346, 561-576). Experiments with genetically modified mice indicate that reducing PDK1 activity to 10% of the normal level is surprisingly well tolerated (M. A. Lawlor et al, EMBO J., 2002, 21, 3728-3738). Certain cancer cells, however, appear to be less able to tolerate antisense-mediated reductions in PDK1 activity (P. Flynn et al, Curr. Biol., 2000, 10, 1439-1442). Moreover, both celecoxib and UCN-01, small molecules that inhibit PDK1 both in vitro and in cells, are capable of inducing apoptosis in cultured tumour cells (S. Arico et al, J. Biol. Chem., 2002, 277, 27613-27621; S. Sato et al. Oncogene, 2002, 21, 1727-1738). Agents that inhibit the PDK1 kinase may therefore be useful for the therapy of cancer.

Chk1

Many standard cancer chemotherapeutic agents act primarily through their ability to induce DNA damage, causing tumour growth inhibition. These agents cause cell cycle arrest, however, by induction of checkpoints at either S-phase or G2-M boundary. The G2 arrest allows the cell time to repair the damaged DNA before entering mitosis. Chk1 and an unrelated serine/threonine kinase, Chk2, play a central role in arresting the cell cycle at the G2-M boundary (M. J. O'Connell et al, EMBO J., 1997, 16, 545-554). Chk1/2 induce this checkpoint by phosphorylating serine 216 of the CDC25 phosphatase, inhibiting the removal of two inactivating phosphates on cyclin dependent kinases (CDKs) (Y. Zeng et al, Nature, 1998, 395, 507-510). Another overlapping pathway mediated by p53 also elicits cycle arrest in response to DNA damage. p53 is mutationally inactivated in many cancers, however, resulting in a partial deficiency in their ability to initiate a DNA repair response. If Chk1 activity is also inhibited in p53-negative cancers, all ability to arrest and repair DNA in response to DNA damage is removed, resulting in mitotic catastrophe and enhancing the effect of the DNA damaging agents (K. Koniaras et al, Oncogene, 2001, 20, 7453-7463; R. T. Bunch et al, Clin. Can. Res., 1996, 2, 791-797; A. Tenzer et al, Curr. Med. Chem., 2003, 3, 35-46). In contrast, normal cells would be relatively unaffected due to retention of a competent p53-mediated cell-cycle arrest pathway. A Chk1 inhibitor (UCN-01) is now in phase I clinical trials for improving the efficacy of current DNA damage inducing chemotherapeutic regimens (E. A. Sausville et al, J. Clinical Oncology, 2001, 19, 2319-2333).

### BRIEF DESCRIPTION OF THE INVENTION

The present invention relates to the use of a class of amino triazolo[1,5-a]pyrimidine compounds as kinase inhibitors, for example CDK2 and/or PDK1 and/or Chk1 inhibitors, for example for inhibition of cancer cell proliferation. A core 7- or 5-amino1,2,4-triazolo[1,5-a]pyrimidine ring with aromatic substitution on the amino group are principle characterising features of the compounds with which the invention is concerned.

### DETAILED DESCRIPTION OF THE INVENTION

According to the present invention there is provided the use of a compound of formula (IA) or (IB) or a salt, N-oxide, hydrate or solvate thereof, in the preparation of a composition for inhibition of kinase activity: 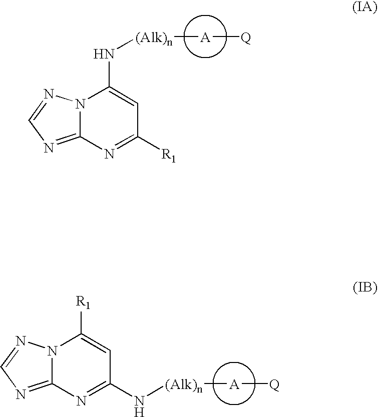
wherein

Ring A is an optionally substituted aryl, heteroaryl, carbocyclic or heterocyclic radical,

Alk represents an optionally substituted divalent C₁-C₆ alkylene radical;

n is 0 or 1;

Q represents a radical of formula -(Alk¹)ₚ-(X)ᵣ-(Alk²)ₛ-Z wherein in any compatible combination
Z is hydrogen or an optionally substituted carbocyclic or heterocyclic ring,
Alk¹ and Alk² are optionally substituted divalent C₁-C₆ alkylene radicals which may contain a —O—, —S— or —NR^{A}— link, wherein R^{A} is hydrogen or C₁-C₆ alkyl,
X represents —O—, —S—, —(C═O)—, —(C═S)—, —SO₂—, —SO—, —C(═O)O—, —OC(═O)—, —C(═O)NR^{A}—, —NR^{A}C(═O)—, —C(═S)NR^{A}—, —NR^{A}C(═S)—, —SO₂NR^{A}, —NR^{A}SO₂—, —OC(═O)NR^{A}—, —NR^{A}C(═O)O—, or —NR^{A}— wherein R^{A} is hydrogen or C₁-C₆ alkyl,
p, r and s are independently 0 or 1, and
R₁ represents a radical -(Cyc)ₖ-(Alk³)ₐ-(Y)_{b}-(Alk⁴)_{d}-B wherein k, a, b and d are independently 0 or 1,
Cyc represents monocyclic divalent carbocyclic or heterocyclic radical having from 5 to 8 ring atoms
Alk³ and Alk⁴ are optionally substituted divalent C₁-C₃ alkylene radicals,
Y represents a monocyclic divalent carbocyclic or heterocyclic radical having from 5 to 8 ring atoms, —O—, —S—, or —NR^{A}— wherein R^{A} is hydrogen or C₁-C₆ alkyl,
B represents hydrogen or halo, or an optionally substituted monocyclic carbocyclic or heterocyclic ring with 5 or 6 ring members, or in the case where Y is —NR^{A}— and b is 1, then R^{A} and the radical -(Alk⁴)_{d}-B taken together with the nitrogen to which they are attached may form an optionally substituted heterocyclic ring.

In particular, the invention relates to the use of such compounds in the preparation of a composition for inhibiting CDK2 and/or PDK1 and/or Chk1 activity.

In another aspect, the invention includes novel compounds of formula (IA) or (IB) as defined and disclosed herein, and salts, hydrates and solvates thereof.

As used herein, the term “(Cₐ-C_{b})alkyl” wherein a and b are integers refers to a straight or branched chain alkyl radical having from a to b carbon atoms. Thus when a is 1 and b is 6, for example, the term includes methyl, ethyl, n-propyl, isopropyl, n-butyl, isobutyl, sec-butyl, t-butyl, n-pentyl and n-hexyl.

As used herein the term “divalent (Cₐ-C_{b})alkylene radical” wherein a and b are integers means a saturated hydrocarbon chain having from a to b carbon atoms and two unsatisfied valences.

As used herein the unqualified term “cycloalkyl” refers to a saturated carbocyclic radical having from 3-8 carbon atoms and includes, for example, cyclopropyl, cyclobutyl, cyclopentyl, cyclohexyl, cycloheptyl and cyclooctyl.

As used herein the term “aryl” refers to a mono-, bi- or tri-cyclic carbocyclic aromatic radical, and to two such radicals covalently linked to each other illustrative of such radicals are phenyl, biphenyl and napthyl.

As used herein the term “carbocyclic” refers to a cyclic radical whose ring atoms are all carbon and to two such cyclic radicals covalently linked to each other, and includes aryl and cycloalkyl radicals.

As used herein the term “heteroaryl” refers to a mono-, bi- or tri-cyclic aromatic radical containing one or more heteroatoms selected from S, N and O. Illustrative of such radicals are thienyl, benzthienyl, furyl, benzfuryl, pyrrolyl, imidazolyl, benzimidazolyl, thiazolyl, benzthiazolyl, isothiazolyl, benzisothiazolyl, pyrazolyl, oxazolyl, benzoxazolyl, isoxazolyl, benzisoxazolyl, isothiazolyl, triazolyl, benztriazolyl, thiadiazolyl, oxadiazolyl, pyridinyl, pyridazinyl, pyrimidinyl, pyrazinyl, triazinyl, indolyl and indazolyl.

As used herein the unqualified term “heterocyclyl” or “heterocyclic” includes “heteroaryl” as defined above, and in particular means a mono-, bi- or tri-cyclic non-aromatic radical containing one or more heteroatoms selected from S, N and O, and to groups consisting of a monocyclic non-aromatic radical containing one or more such heteroatoms which is covalently linked to another such radical or to a monocyclic carbocyclic radical. Illustrative of such radicals are pyrrolyl, furanyl, thienyl, piperidinyl, imidazolyl, oxazolyl, isoxazolyl, thiazolyl, thiadiazolyl, pyrazolyl, pyridinyl, pyrrolidinyl, pyrimidinyl, morpholinyl, piperazinyl, indolyl, morpholinyl, benzfuranyl, pyranyl, isoxazolyl, benzimidazolyl, methylenedioxyphenyl, ethylenedioxyphenyl, maleimido and succinimido groups.

Unless otherwise specified in the context in which it occurs, the term “substituted” as applied to any moiety herein means substituted with at least one substituent, for example selected from (C₁-C₆)alkyl, (C₁-C₆)alkoxy, hydroxy, hydroxy(C₁-C₆)alkyl, mercapto, mercapto(C₁-C₆)alkyl, (C₁-C₆)alkylthio, halo (including fluoro and chloro), trifluoromethyl, trifluoromethoxy, nitro, nitrile (—CN), oxo, phenyl, phenoxy, benzyl, benzyloxy, monocyclic carbocyclic or heterocyclic having from 5 to 7 ring atoms, —COOH, —COOR^{A}, —COR^{A}, —SO₂R^{A}, —CONH₂, —SO₂NH₂, —CONHR^{A}, —SO₂NHR^{A}, —CONR^{A}R^{B}, —SO₂NR^{A}R^{B}, —NH₂, —NHR^{A}, —NR^{A}R^{B}, —OCONH₂, —OCONHR^{A}, —OCONR^{A}R^{B}, —NHCOR^{A}, —NHSO₂R^{A}, —NHCOOR^{A}, —NR^{B}COOR^{A}, —NHSO₂OR^{A}, —NR^{B}SO₂OR^{A}, —NHCONH₂, —NR^{A}CONH₂, —NHCONHR^{B}, —NR^{A}CONHR^{B}, —NHCONR^{A}R^{B}, or —NR^{A}CONR^{A}R^{B} wherein R^{A} and R^{B} are independently a (C₁-C₆)alkyl group. The term “optional substituent” means includes one of the foregoing substituent groups.

As used herein the term “salt” includes base addition, acid addition and quaternary salts. Compounds of the invention which are acidic can form salts, including pharmaceutically or veterinarily acceptable salts, with bases such as alkali metal hydroxides, e.g. sodium and potassium hydroxides; alkaline earth metal hydroxides e.g. calcium, barium and magnesium hydroxides; with organic bases e.g. N-ethyl piperidine, dibenzylamine and the like. Those compounds (I) which are basic can form salts, including pharmaceutically or veterinarily acceptable salts with inorganic acids, e.g. with hydrohalic acids such as hydrochloric or hydrobromic acids, sulphuric acid, nitric acid or phosphoric acid and the like, and with organic acids e.g. with acetic, tartaric, succinic, fumaric, maleic, malic, salicylic, citric, methanesulphonic and p-toluene sulphonic acids and the like.

Some compounds with which the invention is concerned contain one or more actual or potential chiral centres because of the presence of asymmetric carbon atoms. The presence of several asymmetric carbon atoms gives rise to a number of diastereoisomers with R or S stereochemistry at each chiral centre. The invention includes all such diastereoisomers and mixtures thereof.

The Ring A

Ring A is an optionally substituted carbocyclic or heterocyclic radical, preferably monocyclic aryl or heteroaryl radical. Examples of ring A include phenyl, naphthyl, 2-, 3- and 4-pyridyl, 5-pyrimidinyl, 2- and 3-thienyl, 2- and 3-furyl, piperazinyl, pyrrolidinyl, and thiazolinyl. Currently it is preferred that ring A is a phenyl ring.

Ring A may be optionally substituted by any of the substituents listed above in the definition of “optionally substituted”. Examples of optional substituents on ring A or ring B include methyl, ethyl, methylenedioxy, ethylenedioxy, methoxy, ethoxy, methylthio, ethylthio, hydroxy, hydroxymethyl, hydroxyethyl, mercapto, mercaptomethyl, mercaptoethyl, amino, mono- and di-methylamino, mono- and di-ethylamino, fluoro, chloro, bromo, cyano, N-morpholino, N-piperidinyl, N-piperazinyl (the latter being optionally C₁-C₆ alkyl- or benzyl-substituted on the free ring nitrogen).

The Radical -(Alk)ₙ-

When present, the Alk radical acts as a spacer radical between the amino group on the triazolo[1,5-a]pyrimidine ring and the ring A, and may be, for example —CH₂—, —CH₂CH₂—, —CH₂CH(CH₃)—, —CH₂CH₂CH₂—, —CH═CH—, —CH₂CH═CH—, —CH₂CH═CHCH₂—, —CH═CHCH═CH—, —C≡C—, —CH₂C≡C—, or —CH₂C≡CCH₂—. Presently it is preferred that Alk, when present be —CH₂— or —CH₂CH₂—.

However, in another preferred class of compounds with which the invention is concerned, n may be 0 so that the ring A is directly linked to the amino group on the triazolo[1,5a]pyrimidine ring.

The Q Substituent of the Ring A

In the simplest structures with which the invention is concerned, each of p, r and s may be 0, and Z may be hydrogen, so that ring A is simply a carbocyclic or heterocyclic radical, preferably monocyclic aryl or heteroaryl radicalaryl or heteroaryl, optionally substituted as discussed above. A substituent which is presently preferred, when ring A is phenyl, is dimethylsulfonyl especially in the 4-position.

In other simple structures, p, r and s may again each be 0, and Z may be an optionally substituted carbocyclic or heterocyclic ring, for example phenyl, cyclopentyl, cyclohexyl, pyridyl, morpholino, piperidinyl, or piperazyl ring. In such cases, Q is a direct substituent in the optionally substituted ring A.

In more complex structures with which the invention is concerned, one or more of p, r and s may be 1, and Z may be hydrogen or an optionally substituted carbocyclic or heterocyclic ring. For example, p and/or s may be 1 and r may be 0, so that Z is linked to ring A by an alkylene radical, for example a C₁-C₃ alkylene radical, which is optionally substituted. In other cases each of p, r, and s may be 1, in which cases, Z is linked to A by an alkylene radical which is interrupted by the hetero atom-containing X radical. In still other cases, p and s may be 0 and r may be 1, in which case Z is linked to A via the hetero atom-containing X radical. In a preferred example of the latter case, ring A is phenyl, p and s are each 0, X is —SO₂— on the 4-position of the phenyl ring A, and Z is phenyl (optionally substituted).

In one preferred embodiment, p is 0 or 1, r is 1, and X is a sulfonyl —SO₂— radical, a sulfonamide radical —NR^{A}SO₂— or a carboxamide radical —NR^{A}C(═O)— (R^{A} being as defined above, but preferably hydrogen), with the N atom linked to the ring A. In such cases s may be 1 and Z may be hydrogen, so that the group Q is an alkylsulfonyl, alkylsulfonamido or carboxamido substituent in the ring A; or s may be 0 and Q may be an optionally substituted carbocyclic or heterocyclic ring such as optionally substituted phenyl, eg 4-methylphenyl, so that the group Q is an optionally substituted phenylsulfonyl, phenylsulfonamido or carboxamido substituent in the ring A.

The Substituent R₁

R₁ represents a radical -(Cyc)ₖ-(Alk³)ₐ-(Y)_{b}-(Alk⁴)_{d}-B as defined above.

In one class of compounds of the invention k, a, b and d are all 0, and B is hydrogen or halo, so that the pyrimidine ring is either unsubstituted or substituted by halogen, for example chloro or bromo.

In another class of compounds of the invention, B is an optionally substituted monocyclic carbocyclic or heterocyclic ring, for example cyclopentyl, cyclohexyl, phenyl, 2-,3-, or 4-pyridyl, 2-, or 3-thienyl, 2-, or 3-furanyl, pyrrolyl, pyranyl, or piperidinyl ring. Optional substituents in ring B may be any of the substituents listed above in the definition of “optionally substituted”. Examples of optional substituents on ring B include methyl, ethyl, methoxy, ethoxy, methylenedioxy, ethylenedioxy, methylthio, ethylthio, hydroxy, hydroxymethyl, hydroxyethyl, mercapto, mercaptomethyl, mercaptoethyl, amino, mono- and di-methylamino, mono- and di-ethylamino, fluoro, chloro, bromo, cyano, N-morpholino, N-piperidinyl, N-piperazinyl (the latter being optionally C₁-C₆ alkyl- or benzyl-substituted on the free ring nitrogen). Of the foregoing substituents, amino, is currently preferred, particularly when in the 4-position of a cyclohexyl or piperidin-1-yl ring B. In such cases, ring ring B is linked to the pyrimidine ring via linker radical of various types depending on the values of k, a, b and d, and the identities of Cyc, Alk³, Y and Alk⁴. For example, when b and k are 0 and a and/or c is/are 1, the ring B is linked to the pyrimidine ring via an optionally substituted C₁-C₆ alkylene radical; and when k, a and d are 0, b is 1 the ring B may be linked to the pyrimidine ring via an oxygen or sulfur link or via an amino link —NR^{A}- wherein R^{A} is hydrogen or C₁-C₆ alkyl such as methyl or ethyl.

In another class of compounds of the invention k and b are 0, at least one of a and d is 1, and B is hydrogen, so that the pyrimidine ring is substituted by a C₁-C₆ alkyl group, for example methyl, ethyl, and n- or isopropyl, which may itself be substituted by substituents listed above in the definition of “optionally substituted. Examples of optional substituents include methoxy, ethoxy, methylthio, ethylthio, hydroxy, hydroxymethyl, hydroxyethyl, mercapto, mercaptomethyl, mercaptoethyl, amino, mono- and di-methylamino, mono- and di-ethylamino, fluoro, chloro, bromo, and cyano.

In a further class of compounds of the invention k is 0, a is 1 or 0, b is 1, Y is —NR^{A}—, and the radical -(Alk⁴)_{d}-B taken together with R_{A} and the nitrogen to which they are attached form an optionally substituted heterocyclic ring such as a ring piperidinyl, morpholinyl or piperazinyl ring, optionally substituted, for example, by hydroxy, mercapto, methoxy, ethoxy, methylthio, ethylthio, amino, mono- or dimethyl amino, mono- or diethyl amino, nitro, or cyano. In the case of a piperazinyl ring, the second ring nitrogen may optionally be substituted by, for example methyl or ethyl.

In another class of compounds of the invention, k is 1 and Cyc is a phenylene radical, so that the pyrimidine ring is directly substituted by phenyl, which in turn is substituted by -(Alk³)ₐ-(Y)_{b}-(Alk⁴)_{d}-B. In such cases, Y will not normally be a cyclic radical.

Specific examples of R₁ include hydrogen; chloro; phenyl; phenyl substituted by chloro, bromo, hydroxy, amino, methyl; 2- or 3 thienyl; 3,5-dimethylisoxazolylthose present in the compounds of the Examples herein, especially; cyclohexyloxy; cyclopentyloxy; cyclohexylamino; cyclohexylmethyl, piperidin-1-ylmethyl, and cyclohexylamino, all optionally substituted in the cyclohexyl ring by amino, particularly in the 4-position.

Specific compounds with which the invention is concerned include those identified in the Examples.

Novel compounds of formula (I) as discussed also form an aspect of the invention, particularly those wherein n is 0, ring A is phenyl, Q is dimethylaminosulfonyl or phenylsulfonyl, R¹ is 4-aminocyclohexyloxy; 4-aminocyclohexylamino; 4-aminocyclohexylmethyl, or 4-aminopiperidin-1-ylmethyl, and R is chloro, bromo, cyclopentyl, cyclopropyl or isopropyl.

Compounds with which the invention is concerned may be prepared by literature methods, such as those of the preparative Examples herein, and methods analogous thereto.

For example, compounds of the invention wherein R₁ is hydrogen or halo may be prepared by reacting the chloro or dichloro compound (IIA) or (IIB) with the amine (III), 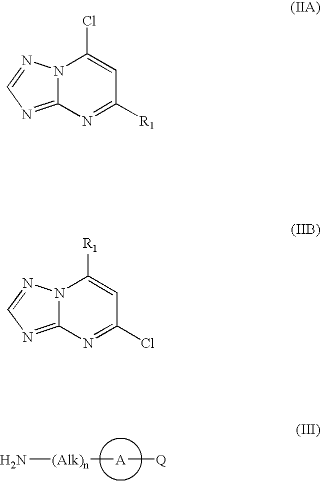
and in the case where R₁ is halo, separating the desired compound (IA) from any resultant contaminant regioisomer (IB), or vice versa.

To prepare compounds of the invention wherein R₁ is a radical —(Y)ₐ—B the general synthetic procedure is based on the coupling of compounds (VA) or (VB), and (VI). 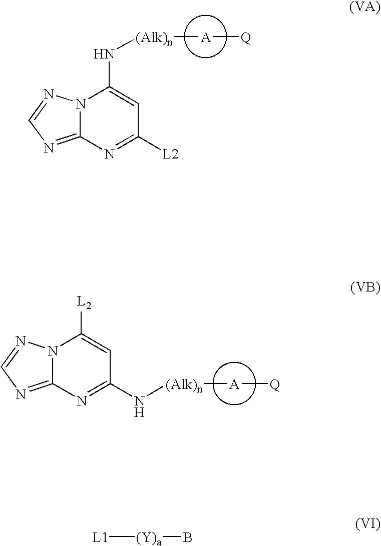
wherein L1 and L2 represent components of a leaving group L1L2.

Thus, to prepare compounds (IA) wherein R₁ is —(Y)ₐ—B wherein a=0 and B is an aryl or heteroaryl ring, a compound of formula (VII) wherein P is an N-protecting group may be reacted with the corresponding aryl or heteroaryl borohydrate compound (VIII) to prepare an intermediate compound (IX), from which the N-protecting group P may be removed to prepare the desired compound (I).

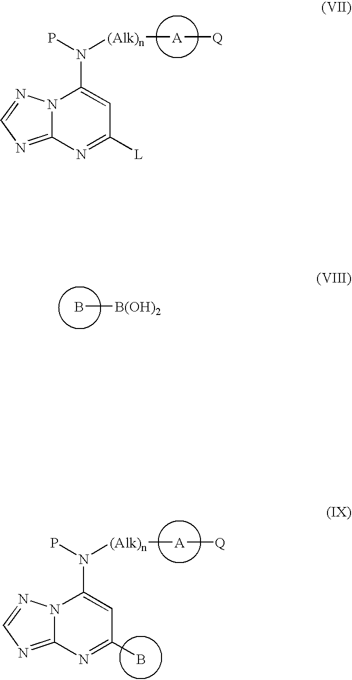

The starting compound (VII) may be prepared by reaction of a compound (V) with an amine (VI) followed by N-protection:

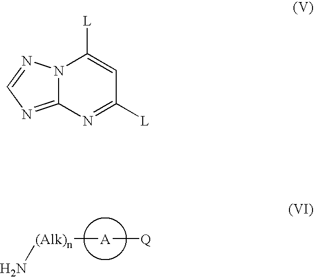

In the above formulae (II)-(VI), L signifies a leaving group such as halo, for example chloro. Rings A and B. Alk, Q and n are as defined in relation to formula (I).

Likewise, to prepare compounds (IA) wherein R₁ is —(Y)ₐ—B wherein a=1, and Y is —O— the compound (VII), where L is chloro, for example, may be reacted with the hydroxy compound HY—B.

Compounds (IB) wherein R₁ is —(Y)ₐ—B wherein a=0 and B is an aryl or heteroaryl ring, or wherein R₁ is —(Y)ₐ—B wherein a=1, and Y is —O—, may be prepared in the same way as the regioisomers (IB), starting from the corresponding regioismers of starting materials (VII).

The compounds with which the invention is concerned are inhibitors of kinases, for example CDK2 and/or PDK1 and/or Chk1, and are thus useful in the treatment of diseases which are mediated by excessive or inappropriate activity of such kinases, CDK2 activity such as cancers, leukemias and other disease states associated with uncontrolled cell proliferation such as psoriasis and restenosis

Accordingly, the invention also provides:

(i) a method of treatment of diseases or conditions mediated by excessive or inappropriate kinase activity, for example CDK2 and/or PDK1 and/or Chk1 activity in mammals, particularly humans, which method comprises administering to the mammal an amount of a compound of formula (IA) or (IB) as defined above, or a salt, hydrate or solvate thereof, effective to inhibit said CDK2 kinase activity and;

(ii) a compound of formula (IA) or (IB) as defined above, or a salt hydrate or solvate thereof, for use in human or veterinary medicine, particularly in the treatment of diseases or conditions mediated by excessive or inappropriate kinase activity, for example CDK2 and/or PDK1 and/or Chk1 activity;

It will be understood that the specific dose level for any particular patient will depend upon a variety of factors including the activity of the specific compound employed, the age, body weight, general health, sex, diet, time of administration, route of administration, rate of excretion, drug combination and the causative mechanism and severity of the particular disease undergoing therapy. In general, a suitable dose for orally administrable formulations will usually be in the range of 0.1 to 3000 mg once, twice or three times per day, or the equivalent daily amount administered by infusion or other routes. However, optimum dose levels and frequency of dosing will be determined by clinical trials as is conventional in the art.

The compounds with which the invention is concerned may be prepared for administration by any route consistent with their pharmacokinetic properties. The orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or colouring agents.

For topical application to the skin, the drug may be made up into a cream, lotion or ointment. Cream or ointment formulations which may be used for the drug are conventional formulations well known in the art, for example as described in standard textbooks of pharmaceutics such as the British Pharmacopoeia.

The active ingredient may also be administered parenterally in a sterile medium. Depending on the vehicle and concentration used, the drug can either be suspended or dissolved in the vehicle. Advantageously, adjuvants such as local anaesthetics, preservatives and buffering agents, can be dissolved in the vehicle.

The following non-limiting Examples illustrate the invention:

In the examples, characterization and/or purification were performed using standard spectroscopic and chromatographic techniques, including liquid chromatography-mass spectroscopy (LC-MS) and high performance liquid chromatography (HPLC), using the conditions described in methods A and B. NMR experiments were conducted on a Bruker DPX400 ultra shield NMR spectrometer in the specified solvent. Reactions carried out under microwave irradiation were conducted in a Smith Synthesizer.

LC-MS Method A
Instrument: HP1100
Column: Luna 3 μm, C18(2), 30 mm×4.6 mm i.d. from Phenomenex
Temperature: 22° C.
Solvents: A—Water+10 mM ammonium acetate+0.08% (v/v) formic acid B—95% Acetonitrile—5% Solvent A+0.08% (v/v) formic acid

Gradient:

| | | | Flow |
|---|---|---|---|
| Time (min) | Solvent A (%) | Solvent B (%) | (cm³min⁻¹) |
| 0 | 95 | 5 | 2 |
| 0.25 | 95 | 5 | 2 |
| 2.50 | 5 | 95 | 2 |
| 2.55 | 5 | 95 | 3 |
| 3.60 | 5 | 95 | 3 |
| 3.65 | 5 | 95 | 3 |
| 3.70 | 5 | 95 | 2 |
| 3.75 | 95 | 5 | 2 |

Detection: UV detection at 230, 254 and 270 nm
Mass Spec: HP1100 MSD, series A Ionization was positive or negative ion electrospray Molecular weight scan range was 120-1000

LC-MS Method B
Instrument: Waters FractionLynx MS autopurification system
Column: Luna 5 μm, C18(2), 100 mm×21.2 mm i.d. from Phenomenex
Temp: ambient
Solvents: A—water+0.08% (v/v) formic acid B—95% methanol-water+0.08% (v/v) formic acid
Flow rate: 20 cm³ Min⁻¹

Gradient:

| Time (min) | Solvent A (%) | Solvent B (%) |
|---|---|---|
| 0 | 95 | 5 |
| 0.5 | 50 | 50 |
| 7.0 | 20 | 80 |
| 7.5 | 5 | 95 |
| 9.5 | 5 | 95 |
| 10.0 | 95 | 5 |

Detection: Photodiode array 210 to 400 nm
Mass spec: MicroMass ZQ Ionization was positive or negative ion electrospray Molecular weight scan range was 150-1000
Collection: Triggered on selected mass ion

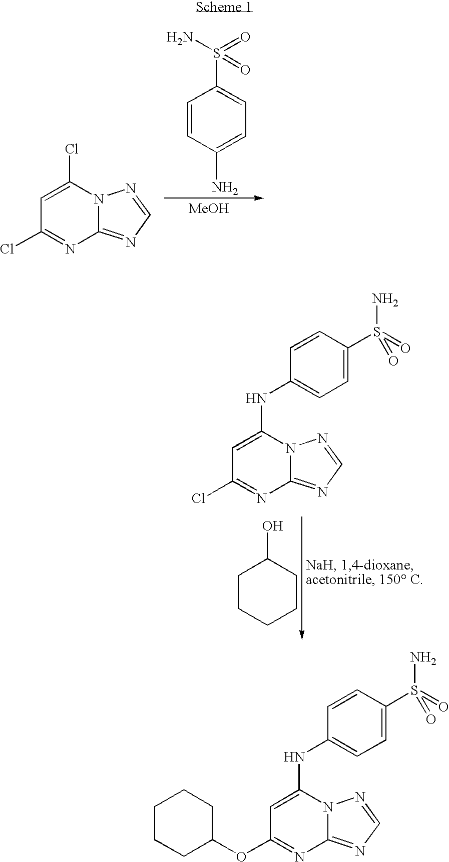

### EXAMPLE 1

### 7-(4-Aminosulfonylphenylamino)-5-chlorotriazolol[1,5-a]pyrimidine

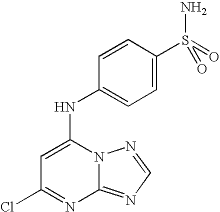

To a solution of 5,7-dichlorotriazolo[1,5-a]pyrimidine (I)¹ (0.3 g, 1.58 mmol) in methanol (4 cm³) was added sulfanilamide (0.273 g, 1.58 mmol). The reaction mixture was stirred at ambient temperature for 4 h. Water (2 cm³) was then added to aid further precipitation. The precipitate was filtered, washed with water and dried in a vacuum oven. The resulting white solid was identified as the title compound (0.38 g, 74%);
1 Y. Makisumi, H. Watanabe and K. Tori, Chem. Pham. Bull., 1964, 12, 204-12.

δ_{H} (400 MHz; d₆-Me₂SO) 10.99 (1 H, s), 8.66 (1 H, s), 7.92-7.89 (2 H, m), 7.68-7.65 (2 H, m), 7.43 (2 H, s) and 6.59 (1 H, s),

m/z 325 and 327 (each MH⁺, 100 and 40%), retention time 1.69 min (method A).

### EXAMPLE 2

### 7-(4-Aminosulfonylphenylamino)-5-cyclohexyloxytriazolo[1,5-a]pyrimidine

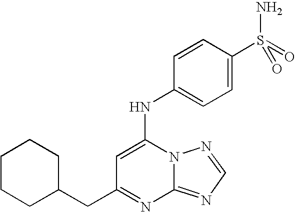

To cyclohexanol (1 cm³, 9.6 mmol) was added sodium hydride (0.072 g, 3 mmol), dioxane (3 cm³), and 7-(4-aminosulfonylphenylamino)-5-chlorotriazolo[1,5-a]pyrimidine (0.15 g, 0.46 mmol). Acetonitrile (0.5 cm³) was then added. The reaction mixture was heated at 150° C. for 10 min in a microwave reactor and then heated for a further period of 10 min. The reaction progress was monitored using LC-MS method A. Further sodium hydride (0.033 g, 1.38 mmol) was added, and the reaction mixture was heated at 150° C. for a further 10 min. A final quantity of sodium hydride (0.033 g, 1.38 mmol) was added, and the reaction mixture was heated at 150° C. for a further 10 min. The reaction mixture was then concentrated in vacuo and the residual material filtered through silica-gel and eluted with methanol. The eluted material, obtained as a yellow solid, was triturated with ethyl acetate. The material was purified by semi-preparative LC-MS (method B), which afforded a yellow solid (0.022 g, 12%), identified as the title compound;

δ_{H} (400 MHz; d₆-Me₂SO) 10.34 (1 H, s), 8.43 (1 H, s), 7.89-7.85 (2 H, m), 7.69-7.62 (2 H, m), 7.38 (1 H, s), 5.93 (1 H, s), 5.11-5.06 (1 H, m), 1.97-1.95 (2 H, m), 1.73-1.70 (2 H, m) and 1.55-1.23 (6 H, m),

m/z 389 (MH⁺, 100%), retention time 2.27 min (method A).

### EXAMPLE 3

### 7-(4-Aminosulfonylphenylamino)-5-(3-methylphenyl)triazolo[1,5-a]pyrimidine

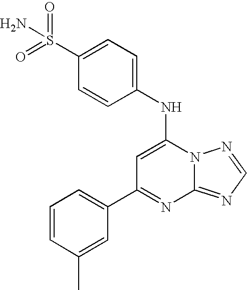

A solution of 7-(4-aminosulfonylphenylamino)-5-chloro[1,2,4]triazolo[1,5-a]pyrimidine (0.078 g, 0.240 mmol), 3-methylphenylboronic acid (0.039 g, 0.288 mmol) and sodium carbonate (0.052 g, 0.504 mmol) in dioxane (1 cm³) and water (1 cm³) was placed in a reaction tube and degassed with a stream of nitrogen for 10 min. Tetrakis(triphenylphosphine)palladium(0) (0.056 g, 0.048 mmol) was then added, the reaction-tube sealed and then irradiated with microwaves for 20 min whilst the temperature was maintained at 150° C. The reaction mixture was then cooled and evaporated. The residue was subjected to silica-gel column chromatography [CH₂Cl₂ then CH₂Cl₂-MeOH (9:1) as eluent] which afforded one major fraction. The eluted material, obtained as a white solid (0.027 g, 30%), was identified as the title compound;

δ_{H} (400 MHz; d₆-Me₂SO) 10.69 (1 H, br s), 8.64 (1 H, s), 7.94 (1 H, s), 7.91 (2 H, d, J 8.5 Hz), 7.86 (1 H, d, J 7.7 Hz), 7.74 (2 H, d, J 8.5 Hz), 7.43-7.39 (3 H, m), 7.34 (1 H, d, J 7.5 Hz), 7.13 (1 H, s) and 2.40 (3 H, s),

m/z 381 (MH⁺, 100%), retention time 2.02 min (Method A).

### EXAMPLE 4

### 4-(5-Diethylamino-[1,2,4]triazolo[1,5-a]pyrimidine-7-ylamino)-benzenesulfonamide

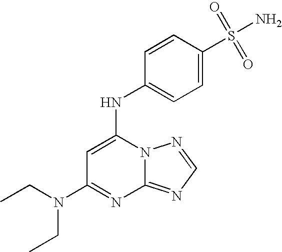

To a suspension of 4-(5-chloro-[1,2,4]triazolo[1,5-a]pyrimidine-7-ylamino)-benzenesulfonamide (0.075 g 0.23 mmol) in ethanol (3 cm³) was added diethylamine (0.036 cm³ 0.35 mmol). The reaction mixture was sealed in a Smith process vial and heated under microwave irradiation to 150° C. for 600 s, cooled and then reheated to 160° C. for a further 1200 s. The cooled reaction mixture was concentrated in vacuo and the residue was purified by semi-preparative LC-MS (Method B), which afforded a white solid (0.026 g, 31%);

δ_{H} (400 MHz; d₆-Me₂SO) 9.85 (1 H, br s), 8.25 (1 H, s), 7.90 (2 H, d J 8 Hz) 7.65 (2 H, d, J 8 Hz), 7.40 (2 H, br s), 6.00 (1 H, s), 3.55 (4 H, br d, J 7 Hz), 1.20 (6 H, t, J 7 Hz),

m/z 362 (MH⁺, 100%), retention time 1.83 min (Method A).

Examples 5-103 in the following Tables 1 and 2 were prepared by methods analogous to Examples 1-4 above. The compounds of Examples 1-4 are also included. Most of the compounds were tested for activity in at least one of the assays described below in the Assay section. The result obtained in each case is given in the Tables 1 and 2.

TABLE 1

| | | | | | | | RT |
|---|---|---|---|---|---|---|---|
| | Method | | | | | | (min) |
| | of | | CDK2 | PDK1 | Chk1 | | (LC-MS |
| | Example | Example | IC₅₀ | IC₅₀ | IC₅₀ | | Method |
| Structure | No | no | (μM) | (μM) | (μM) | m/z | A) |
| 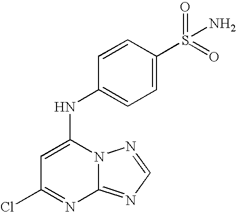 | 1 | 1 | 20.07 | >200 | >200 | 325 and 327 (MH⁺, 100 and 35%) | 1.70 |
| 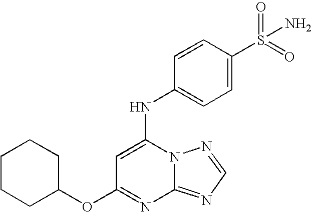 | 2 | 2 | 0.34 | >200 | >200 | 389 (MH⁺, 100%) | 2.26 |
| 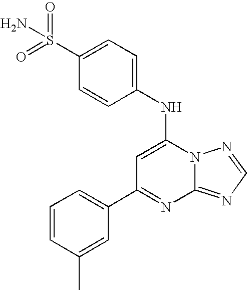 | 3 | 3 | 1.82 | >200 | >200 | 381 (MH⁺, 100%) | 2.03 |
| 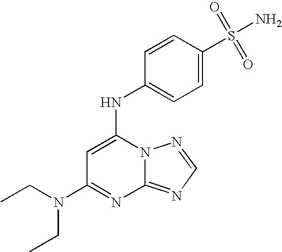 | 4 | 4 | 0.39 | >200 | >200 | 362 (MH⁺, 100%) | 1.85 |
| 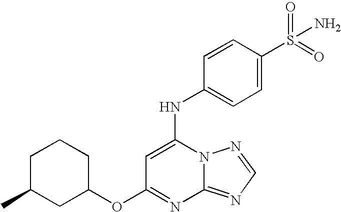 | 2 | 5 | 6.40 | >200 | >200 | 403 (MH⁺, 100%) | 2.37 |
| 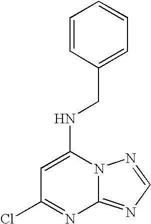 | 1 | 6 | 84.98 | >200 | >200 | 262 and 260 (each MH⁺, 100 and 35%) | 2.14 |
| 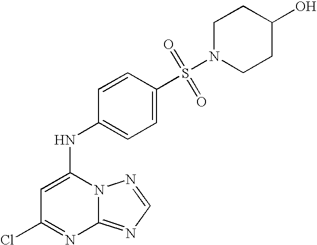 | 1 | 7 | 22.70 | >200 | >200 | 409 and 411 (each MH⁺, 100 and 35%) | 1.87 |
| 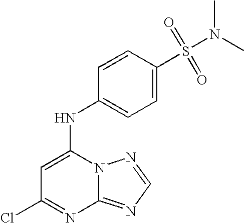 | 1 | 8 | 48.20 | >200 | >200 | 353 and 355 (each MH⁺, 100 and 35%) | 2.05 |
| 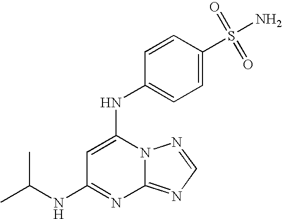 | 4 | 9 | 0.89 | >200 | >200 | 348 (MH⁺, 100%) | 1.64 |
| 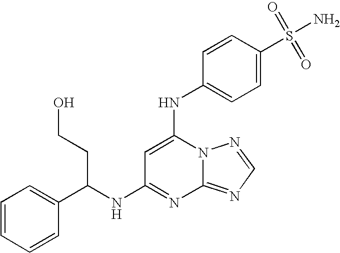 | 4 | 10 | 5.74 | >200 | >200 | 440 (MH⁺, 100%) | 1.72 |
| 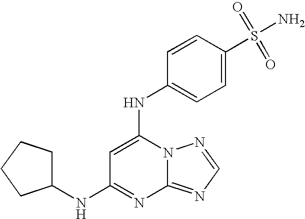 | 4 | 11 | 0.55 | >200 | >200 | 374 (MH⁺, 100%) | 1.81 |
| 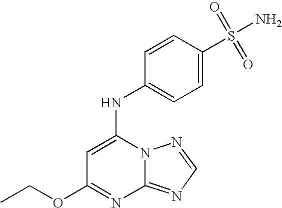 | 2 | 12 | 13.03 | >200 | >200 | 335 (MH⁺, 100%) | 1.81 |
| 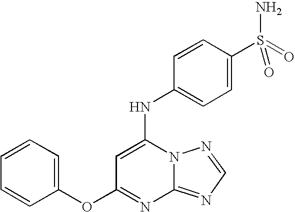 | 2 | 13 | 20.11 | >200 | >200 | 383 (MH⁺, 100%) | 1.90 |
| 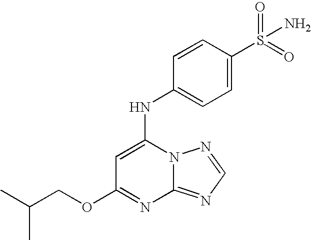 | 2 | 14 | 1.85 | >200 | >200 | 363 (MH⁺, 100%) | 1.97 |
| 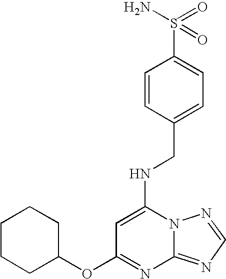 | 2 | 15 | 11.25 | >200 | >200 | 403 (MH⁺, 100%) | 2.23 |
| 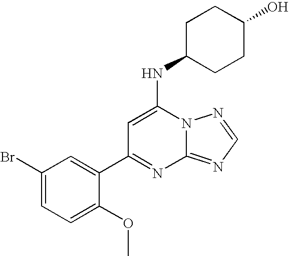 | 3 | 16 | 48.03 | >200 | >200 | 418 and 420 (each MH⁺, 100%) | 2.07 |
| 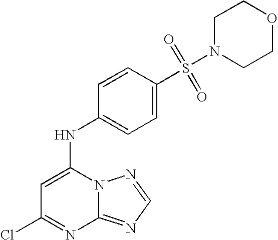 | 1 | 17 | 16.71 | >200 | >200 | 395 and 397 (each MH⁺, 100 and 35%) | |
| 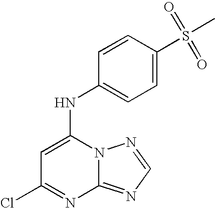 | 1 | 18 | 0.82 | >200 | >200 | 324 and 326 (each MH⁺, 100 and 35%) | |
| 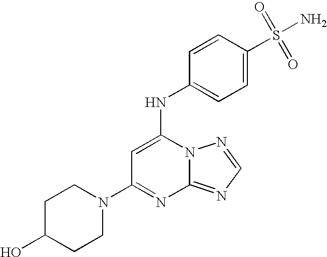 | 4 | 19 | 0.99 | >200 | >200 | 390 (MH⁺, 100%) | 1.54 |
| 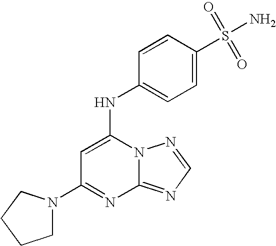 | 4 | 20 | 3.33 | >200 | >200 | 360 (MH⁺, 100%) | 1.75 |
| 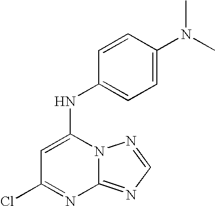 | 1 | 21 | 58.99 | >200 | >200 | 289 and 291 (each MH⁺, 100 and 35%) | 2.22 |
| 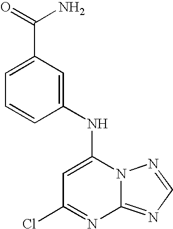 | 1 | 22 | 36.66 | >200 | >200 | 289 and 291 (each MH⁺, 100 and 35%) | 2.40 |
| 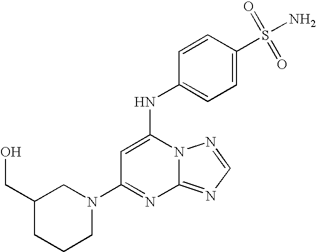 | 4 | 23 | 0.33 | >200 | >200 | 404 (MH⁺, 100%) | 1.68 |
| 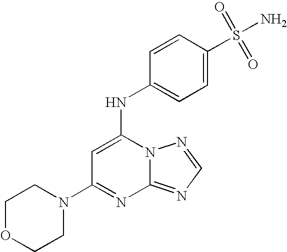 | 4 | 24 | 15.70 | >200 | >200 | 376 (MH⁺, 100%) | 1.65 |
| 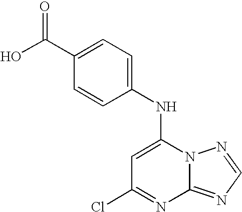 | 1 | 25 | 93.19 | >200 | >200 | 290 and 292 (each MH⁺, 100 and 35%) | 1.75 |
| 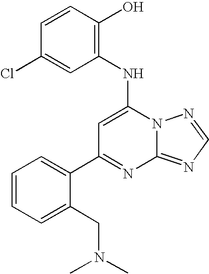 | 3 | 26 | >200 | >200 | 105.00 | 395 and 397 (each MH⁺, 100 and 35%) | 1.85 |
| 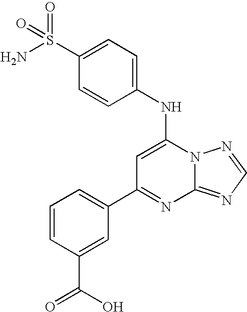 | 3 | 27 | 14.28 | >200 | >200 | 411 (MH⁺, 100%) | 1.73 |
| 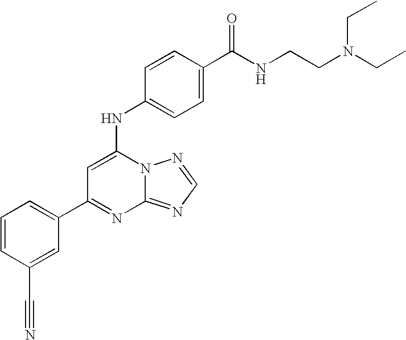 | 3 | 28 | 18.58 | >200 | >200 | 455 (MH⁺, 100%) | 1.71 |
| 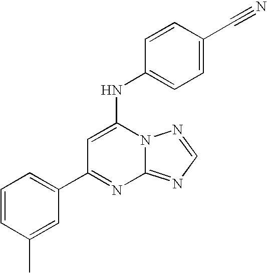 | 3 | 29 | 57.45 | >200 | >200 | 327 (MH⁺, 100%) | 2.36 |
| 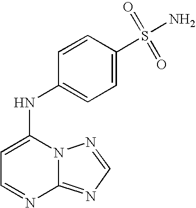 | By hydro- genation of com- pound of Example 1 | 30 | 33.03 | >200 | 71.99 | 291 (MH⁺, 100%) | 1.44 |
| 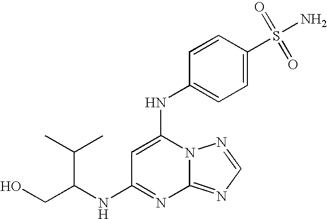 | 4 | 31 | 4.91 | >200 | >200 | 392 (MH⁺, 100%) | 1.71 |
| 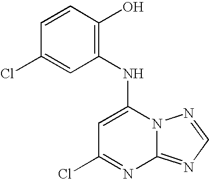 | 1 | 32 | 31.59 | >200 | >200 | 296, 298 and 300 (each MH⁺, 90, 60 and 10%) | 2.09 |
| 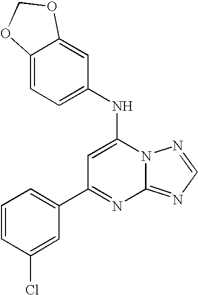 | 3 | 33 | 28.96 | >200 | 23.87 | 366 and 368 (each MH⁺, 100 and 35%) | 2.45 |
| 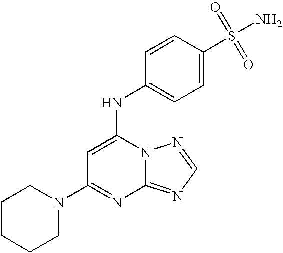 | 4 | 34 | 0.62 | >200 | >200 | 374 (MH⁺, 100%) | 1.89 |
| 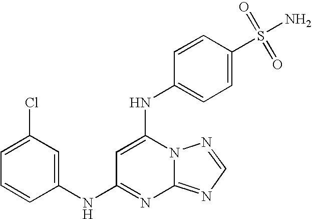 | 4 | 35 | 1.72 | 47.98 | 40.46 | 416 and 418 (each MH⁺, 100%) | 2.19 |
| 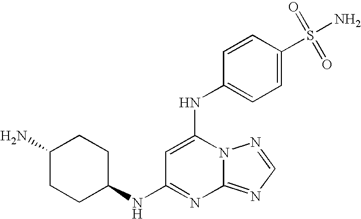 | 4 | 36 | 0.27 | 43.32 | 93.45 | 403 (MH⁺, 100%) | 1.37 |
| 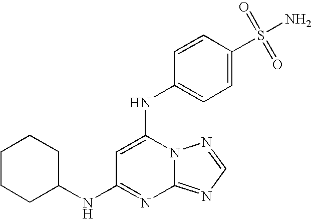 | 4 | 37 | 0.73 | 63.95 | >200 | 388 (MH⁺, 100%) | 2.05 |
| 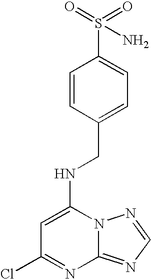 | 1 | 38 | 15.03 | >200 | >200 | 339 and 341 (each MH⁺, 100 and 35%) | 1.71 |
| 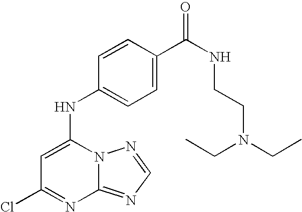 | 1 | 39 | 42.81 | >200 | >200 | 388 and 390 (each MH⁺, 100 and 35%) | 1.48 |
| 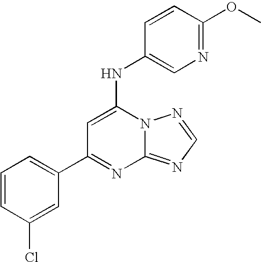 | 3 | 40 | 8.29 | >200 | >200 | 353 (MH⁺, 100%) | 2.43 |
| 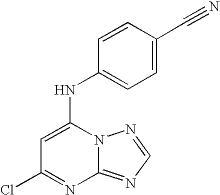 | 1 | 41 | 61.37 | >200 | >200 | 271 and 273 (each MH⁺, 100 and 35%) | 2.01 |
| 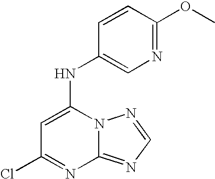 | 1 | 42 | 11.78 | >200 | >200 | 277 and 279 (each MH⁺, 100 and 35%) | 1.94 |
| 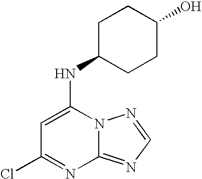 | 1 | 43 | >200 | >200 | >200 | 268 and 270 (each MH⁺, 100 and 35%) | 1.56 |
| 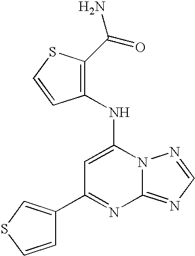 | 3 | 44 | 60.32 | >200 | >200 | 343 (MH⁺, 100%) | 2.02 |
| 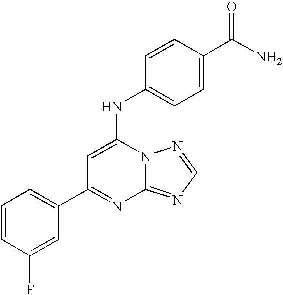 | 3 | 45 | 12.29 | >200 | >200 | 349 (MH⁺, 100%) | 1.92 |
| 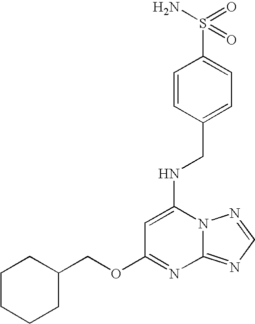 | 2 | 46 | 18.72 | >200 | >200 | 349 (MH⁺, 100%) | 2.39 |
| 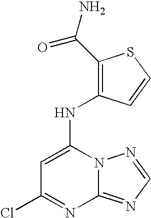 | 1 | 47 | >200 | >200 | >200 | 295 and 297 (each MH⁺, 100 and 35%) | 1.78 |
| 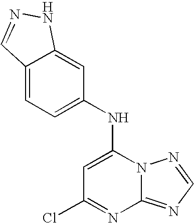 | 1 | 48 | 13.47 | >200 | >200 | 286 and 288 (each MH⁺, 100 and 35%) | 1.82 |
| 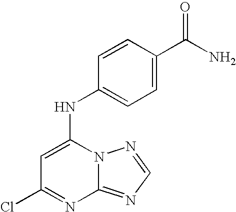 | 1 | 49 | 24.13 | >200 | >200 | 289 and 291 (each MH⁺, 100 and 35%) | |
| 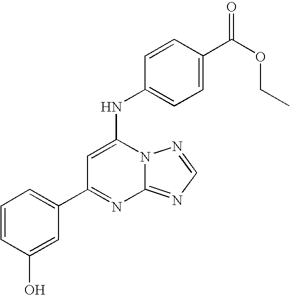 | 3 | 50 | 31.91 | >200 | >200 | 376 (MH⁺, 100%) | 2.27 |
| 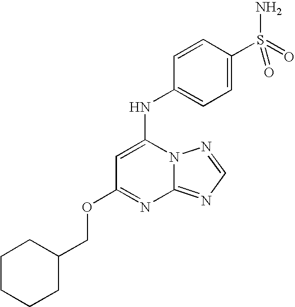 | 2 | 51 | 11.20 | >200 | >200 | 403 (MH⁺, 100%) | |
| 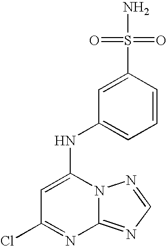 | 1 | 52 | 40.86 | >200 | >200 | 325 and 327 (each MH⁺, 100 and 35%) | 1.69 |
| 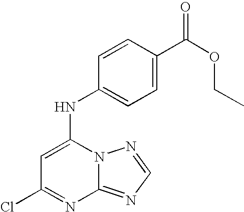 | 1 | 53 | >200 | >200 | >200 | 318 and 320 (each MH⁺, 100 and 35%) | 2.28 |
| 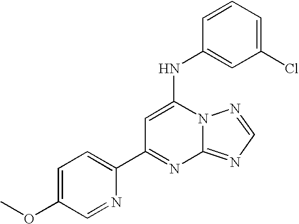 | 3 | 54 | >200 | >200 | >200 | 353 (MH⁺, 100%) | 2.44 |
| 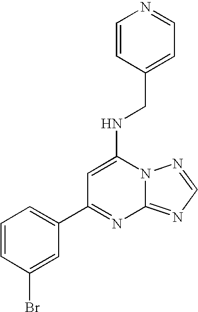 | 3 | 55 | 26.30 | >200 | >200 | 381 and 383 (each MH⁺, 100%) | 1.94 |
| 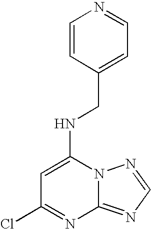 | 1 | 56 | 43.25 | >200 | >200 | 261 and 263 (each MH⁺, 100 and 35%) | 1.23 |

TABLE 2

| | | | | | | | RT |
|---|---|---|---|---|---|---|---|
| | Method | | | | | | (min) |
| | of | | CDK2 | Chk1 | PDK1 | | (LC-MS |
| | Example | Example | IC₅₀ | IC₅₀ | IC₅₀ | | Method |
| Structure | No | no | (μM) | (μM) | (μM) | m/z | A) |
| 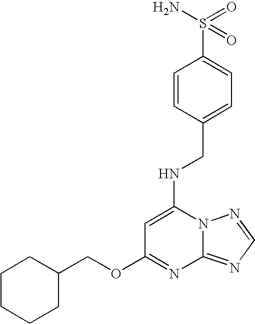 | 2 | 57 | 18.72 | >200 | >200 | 417 (MH⁺, 100%) | 2.39 |
| 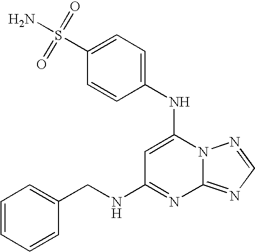 | 4 | 58 | 13.61 | >200 | >200 | 396 (MH⁺, 100%) | 1.96 |
| 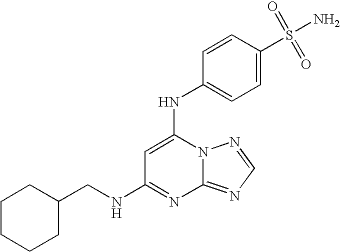 | 4 | 59 | 3.86 | 117.25 | 118.3 | 402 (MH⁺, 100%) | 2.16 |
| 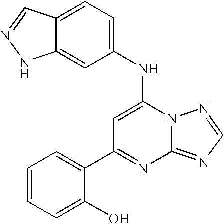 | 3 | 60 | 1.91 | >200 | >200 | 344 (MH⁺, 100%) | 2.12 |
| 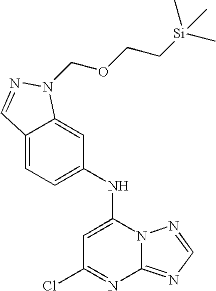 | 1 | 61 | >200 | >200 | >200 | 416 and 418 (each MH⁺, 100 and 35%) | 2.54 and 2.59 |
| 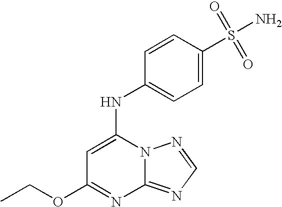 | 2 | 62 | 13.03 | >200 | >200 | 335 (MH⁺, 100%) | 1.82 |
| 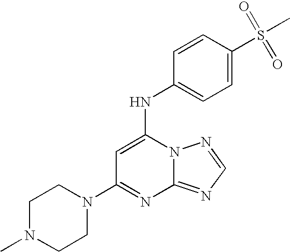 | 4 | 63 | 46.21 | >200 | >200 | 388 (MH⁺, 100%) | |
| 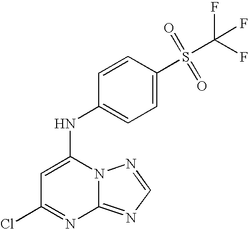 | 1 | 64 | 154.58 | >200 | >200 | 378 and 380 (each MH⁺, 100 and 35%) | 2.39 |
| 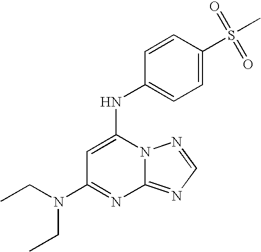 | 4 | 65 | 0.94 | >200 | >200 | 361 (MH⁺, 100%) | 1.96 |
| 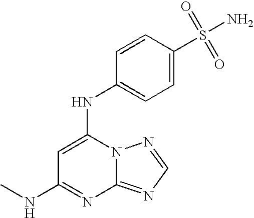 | 4 | 66 | 4.19 | >200 | >200 | 320 (MH⁺, 100%) | 1.52 |
| 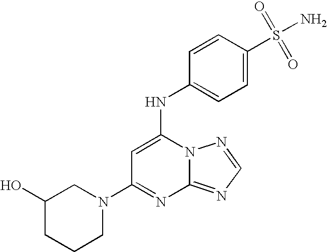 | 4 | 67 | 2.30 | >200 | >200 | 390 (MH⁺, 100%) | 1.56 |
| 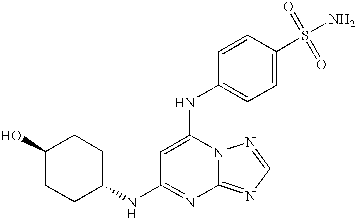 | 4 | 68 | 7.40 | >200 | >200 | 404 (MH⁺, 100%) | 1.56 |
| 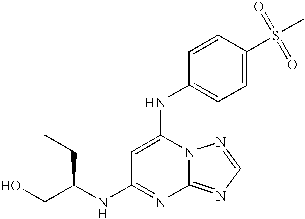 | 4 | 69 | 1.82 | >200 | >200 | 377 (MH⁺, 100%) | 1.72 |
| 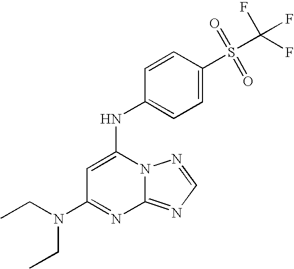 | 4 | 70 | 2.69 | >200 | >200 | 415 (MH⁺, 100%) | 2.52 |
| 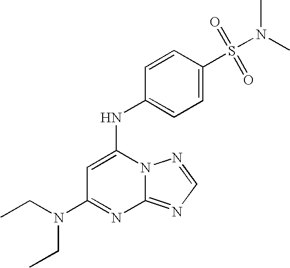 | 4 | 71 | 0.59 | >200 | >200 | 390 (MH⁺, 100%) | 2.17 |
| 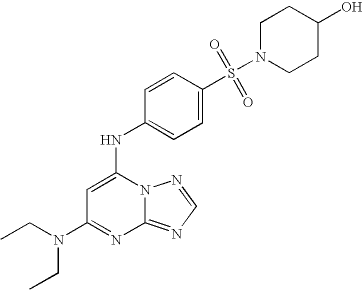 | 4 | 72 | 0.38 | >200 | >200 | 446 (MH⁺, 100%) | 1.44 |
| 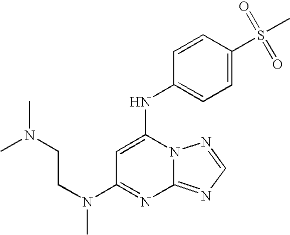 | 4 | 73 | 22.49 | >200 | >200 | 390 (MH⁺, 100%) | 1.44 |
| 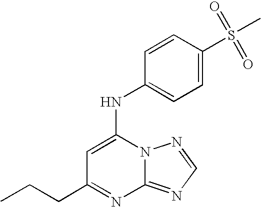 | 1 (starting from 7- chloro- 5-propyl triazolo [1,5-a]pyrimidine) | 74 | 14.25 | >200 | >200 | 332 (MH⁺, 100%) | 1.90 |
| 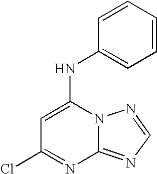 | 1 | 75 | 62.76 | >200 | >200 | 246 and 248 (each MH⁺, 100 and 35%) | 2.11 |
| 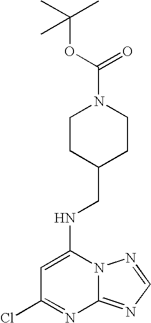 | 1 | 76 | >200 | >200 | >200 | 367 and 369 (each MH⁺, 100 and 35%) | 2.31 |
| 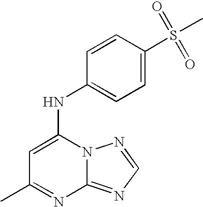 | 1 (starting from 7- chloro- 5-methyl triazolo [1,5-a]pyrimidine) | 77 | 124.50 | >200 | >200 | 304 (MH⁺, 100%) | 1.64 |
| 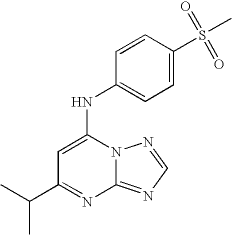 | 1 (starting from 7- chloro- 5-isopropyl triazolo [1,5-a]pyrimidine) | 78 | 7.42 | >200 | >200 | 332 (MH⁺, 100%) | 1.91 |
| 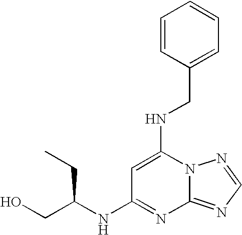 | 4 | 79 | 5.05 | >200 | >200 | 313 (MH⁺, 100%) | 1.89 |
| 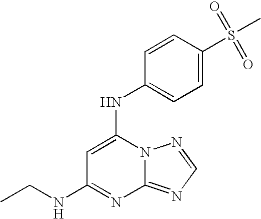 | 4 | 80 | 13.46 | >200 | >200 | 333 (MH⁺, 100%) | 1.73 |
| 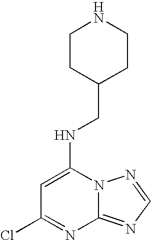 | 1 | 81 | 177.31 | >200 | >200 | 267 (MH⁺, 100%) | 1.07 |
| 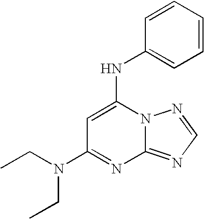 | 4 | 82 | 4.66 | >200 | >200 | 283 (MH⁺, 100%) | 2.26 |
| 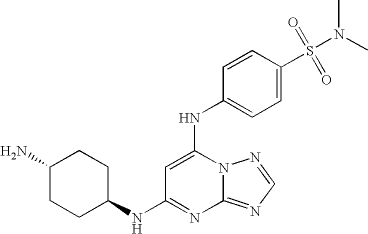 | 4 | 83 | 0.27 | 93.45 | 43.32 | 431 (MH⁺, 100%) | 1.61 |
| 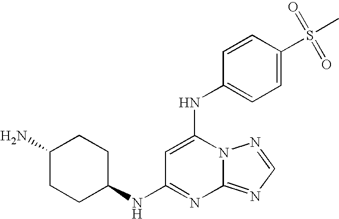 | 4 | 84 | 0.25 | >200 | 107.3 | 402 (MH⁺, 100%) | 1.45 |
| 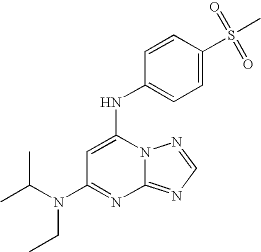 | 4 | 85 | 0.86 | >200 | 2.889 | 375 (MH⁺, 100%) | 2.06 |
| 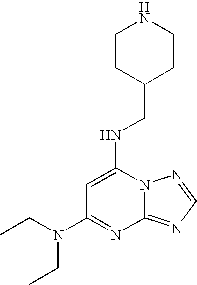 | 4 | 86 | — | >200 | >200 | 304 (MH⁺, 100%) | 1.40 |
| 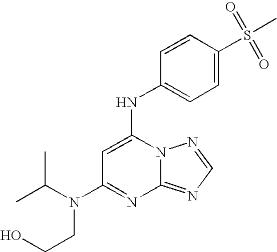 | 4 | 87 | 1.49 | >200 | >200 | 391 (MH⁺, 100%) | 1.78 |
| 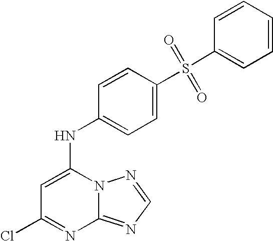 | 1 | 88 | 5.98 | 11.89 | >200 | 386 and 388 (each MH⁺, 100 and 35%) | 2.23 |
| 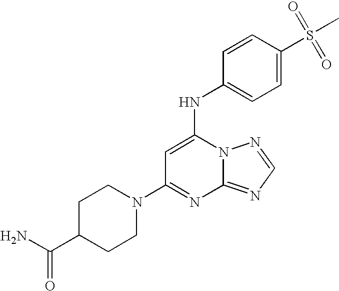 | 4 | 89 | 36.04 | >200 | >200 | 416 (MH⁺, 100%) | 1.59 |
| 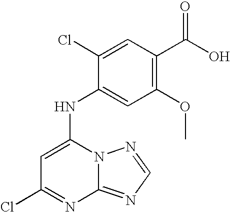 | 1 | 90 | 101.77 | >200 | >200 | 354, 356 and 358 (each MH⁺, 90, 60 and 10%) | 1.77 |
| 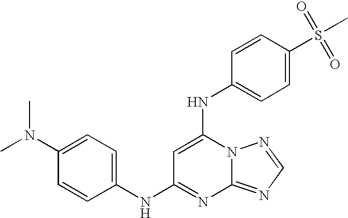 | 4 | 91 | 4.83 | 14.19 | >200 | 424 (MH⁺, 100%) | 1.85 |
| 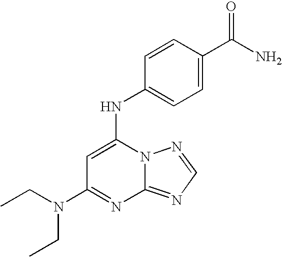 | 4 | 92 | 1.49 | >200 | >200 | 326 (MH⁺, 100%) | 1.73 |
| 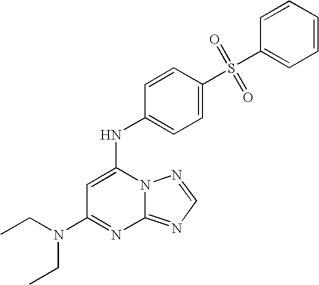 | 4 | 93 | 0.67 | >200 | >200 | 423 (MH⁺, 100%) | 2.35 |
| 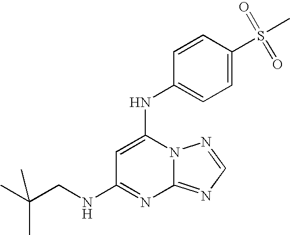 | 4 | 94 | 1.53 | >200 | >200 | 375 (MH⁺, 100%) | 2.13 |
| 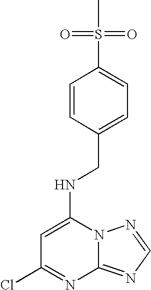 | 1 | 95 | 76.19 | >200 | >200 | 338 and 340 (each MH⁺, 100 and 35%) | 1.80 |
| 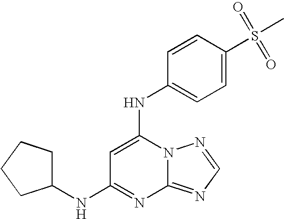 | 4 | 96 | 2.23 | >200 | >200 | 373 (MH⁺, 100%) | 2.05 |
| 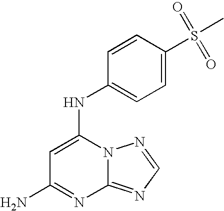 | 4 | 97 | 20.32 | >200 | >200 | 305 (MH⁺, 100%) | 1.54 |
| 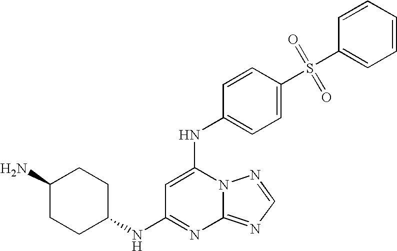 | 4 | 98 | 0.26 | 2.19 | 14.22 | 464 (MH⁺, 100%) | 1.78 |
| 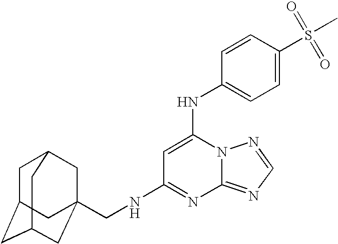 | 4 | 99 | 25.69 | >200 | >200 | 453 (MH⁺, 100%) | 2.54 |
| 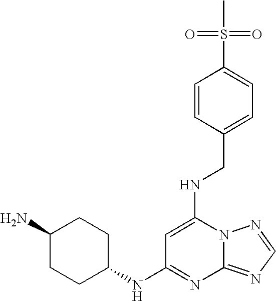 | 4 | 100 | 2.95 | >200 | >200 | 416 (MH⁺, 100%) | 1.41 |
| 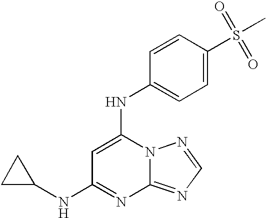 | 4 | 101 | 15.07 | >200 | >200 | 345 (MH⁺, 100%) | 1.79 |
| 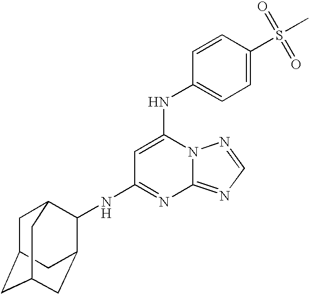 | 4 | 102 | 10.92 | 59.35 | 3.55 | 439 (MH⁺, 100%) | 2.47 |
| 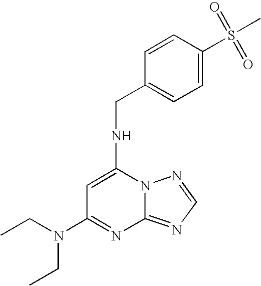 | 4 | 103 | 29.81 | >200 | >200 | 375 (MH⁺, 100%) | 1.89 |

### EXAMPLE 104

### 7-(3-Methylphenyl)-5-cholorotriazolo[1,5-a]pyrimidine

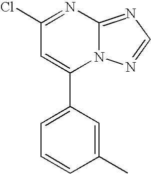

To a solution of 5,7dichlorotriazolo[1,5-a]pyrimidine¹ (0.50 g, 2.65 mmol) in anhydrous tetrahydrofuran (10 cm³) under an atmosphere of nitrogen was added a 1M solution of 3-tolylmagnesium chloride in tetrahydrofuran (3.2 cm³, 3.18 mmol). The reaction mixture was stirred at ambient temperature for 1 h. The reaction was then quenched with 2M hydrochloric acid (3 cm³), extracted with dichloromethane (30 cm³) and filtered through an hydrophobic frit. The reaction mixture was then concentrated in vacuo and the residual material filtered through silica-gel and eluted with dichloromethane. The eluted material was concentrated in vacuo, affording a white solid (0.51 g, 78%), identified as the title compound;
1. Y. Makisumi, H. Watanabe and K. Tori, Chem. Pharm. Bull., 1964, 12, 204-12.

δ_{H} (400 MHz; d6-Me₂SO) 8.55 (1 H, s), 7.80 (2 H, m) 7.6 (1 H, s), 7.35 (2H, d) and 2.3 (3H, s).

m/z 245, 247 and 246 (each MH⁺, 100, 37 and 19%), retention time 2.38 min (method A).

### 4-[7-(3-Methylphenyl)]-[1,2,4]triazolo[1,5-a]pyrimidin-5-ylamino)-benzamide

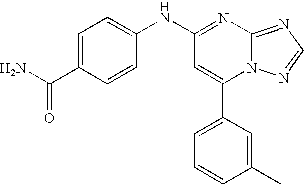

4-Aminobenzamide (0.031 g, 0.22 mmol) was added to a solution of 7-(3-methylphenyl)-5-chlorotriazolo[1,5-a]pyrimidine (0.050 g, 0.20 mmol) in ethyl alcohol (2 cm³). The reaction mixture was heated to 150° C. for 10 min in a microwave reactor. The reaction mixture was then cooled and the formed precipitate collected by filtration. The precipitate was then purified by semi-preparative LCMS (method B), affording a white solid (0.017 g, 24%), identified as the title compound;

δ_{H} (400 MHz; d₆-Me₂SO) 10.40 (1 H, s), 8.50 (1 H, s), 8.05 (4 H, s), 7.99 (1 H, d), 7.90 (1 H, d), 7.6 (1 H, t), 7.55 (1 H, d), 7.35 (1 H, br s), 7.0 (1 H, s) and 2.5 (3 H, s).

m/z 345 and 346 (MH⁺, 100 and 40%), retention time 2.05 min (method A).

Examples 104-167 in the following Tables 3 and 4 were prepared by methods analogous to Examples 104 above. The compound of Example 104 is also included. All compounds were tested for activity in at least one of the assays described below in the Assay section. The result obtained in each case is given in the Tables 3 and 4.

TABLE 3

| | | | | | | RT |
|---|---|---|---|---|---|---|
| | | | | | | (min) |
| | | PDK1 | CDK2 | Chk1 | | (LC-MS |
| | Example | IC₅₀ | IC₅₀ | IC₅₀ | | Method |
| Structure | No | (μM) | (μM) | (μM) | m/z | A) |
| 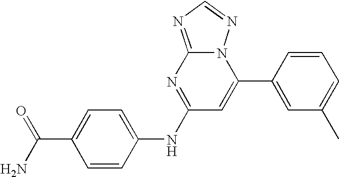 | 104 | 4.08 | >200 | 14.86 | 345 (MH⁺, 100) | 2.05 |
| 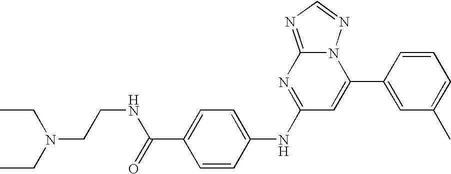 | 105 | 6.10 | >200 | 5.45 | 444 (MH⁺, 100%) | 1.90 |
| 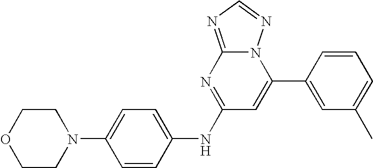 | 106 | 23.22 | >200 | 63.33 | 387 (MH⁺, 100%) | 2.36 |
| 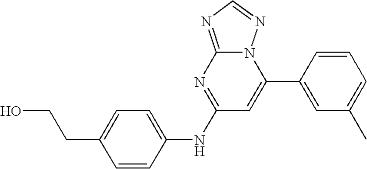 | 107 | 14.67 | >200 | 42.21 | 346 (MH⁺, 100%) | 2.19 |
| 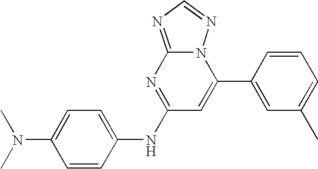 | 108 | 66.84 | >200 | 36.83 | 345 (MH⁺, 100%) | 2.24 |
| 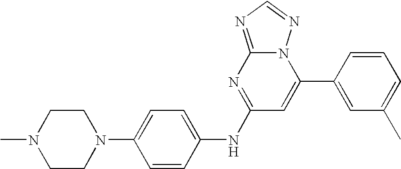 | 109 | 36.00 | >200 | 6.70 | 400 (MH⁺, 100%) | 1.84 |
| 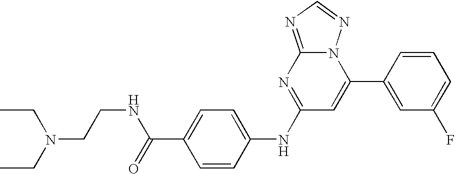 | 110 | 9.33 | 14.11 | 0.85 | 388 (MH⁺, 100%) | 1.83 |
| 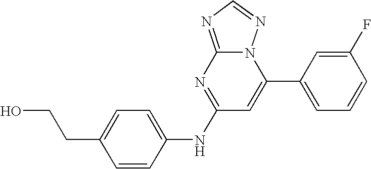 | 111 | 52.21 | >200 | 9.57 | 350 (MH⁺, 100%) | 2.13 |
| 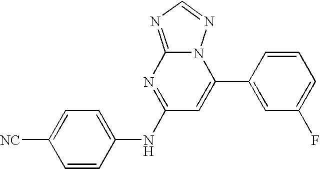 | 112 | 51.64 | 119.19 | >200 | 331 (MH⁺, 100%) | 2.37 |
| 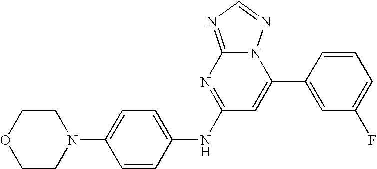 | 113 | 46.49 | >200 | 5.62 | 391 (MH⁺, 100%) | 2.26 |
| 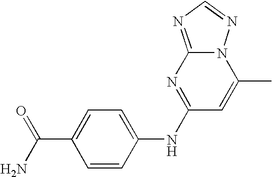 | 114 | >200 | >200 | 6.42 | 269 (MH⁺, 100%) | 1.53 |
| 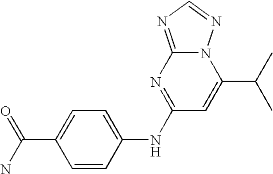 | 115 | >200 | 147.59 | 83.73 | 297 (MH⁺, 100%) | 1.84 |
| 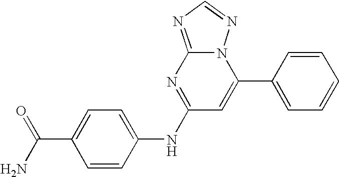 | 116 | 5.11 | 97.55 | 9.82 | 331 (MH⁺, 100%) | 1.94 |
| 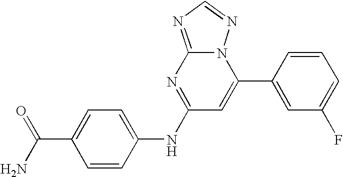 | 117 | 7.30 | >200 | 3.05 | 349 (MH⁺, 100%) | 1.97 |
| 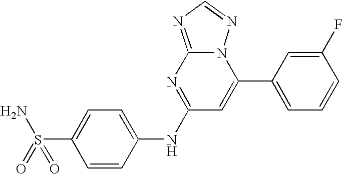 | 118 | 6.28 | 116.36 | 1.54 | 385 (MH⁺, 100%) | 2.10 |
| 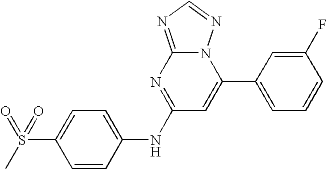 | 119 | 114.77 | >200 | 2.58 | 384 (MH⁺, 100%) | 2.14 |
| 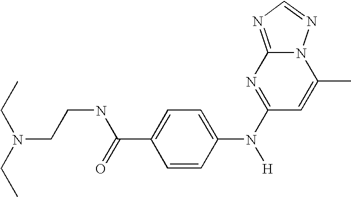 | 120 | >200 | >200 | 0.82 | 368 (MH⁺, 100%) | 1.49 |
| 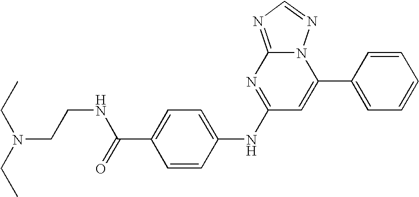 | 121 | 15.43 | >200 | 2.25 | 430 (MH⁺, 100%) | 1.78 |
| 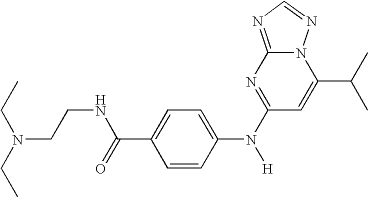 | 122 | >200 | >200 | 22.93 | 396 (MH⁺, 100%) | 1.69 |
| 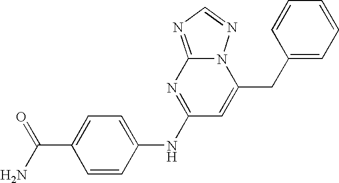 | 123 | >200 | >200 | 31.42 | 345 (MH⁺, 100%) | 1.99 |
| 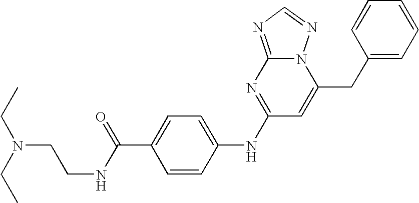 | 124 | >200 | >200 | 15.82 | 444 (MH⁺, 100%) | 1.86 |
| 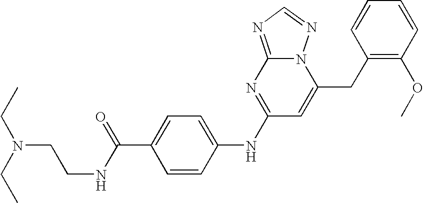 | 125 | >200 | >200 | 37.35 | 474 (MH⁺, 100%) | 1.90 |
| 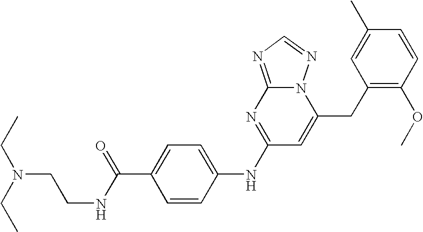 | 126 | 68.14 | >200 | 26.85 | 499 (MH⁺, 100%) | 1.83 |
| 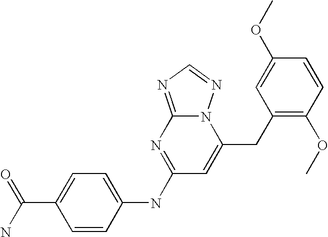 | 127 | >200 | >200 | 106.02 | 405 (MH⁺, 100%) | 2.07 |
| 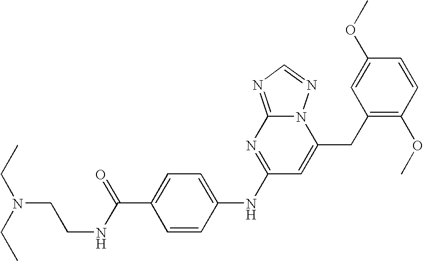 | 128 | >200 | >200 | 65.85 | 504 (MH⁺, 100%) | 1.89 |
| 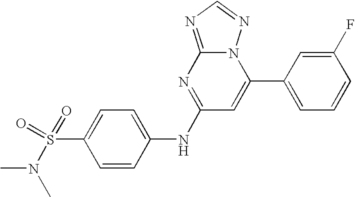 | 129 | 30.60 | >200 | 12.17 | 413 (MH⁺, 100%) | 2.35 |
| 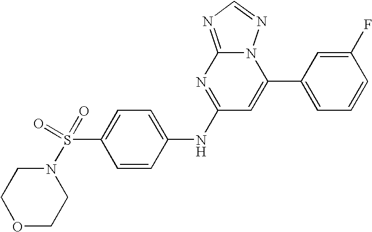 | 130 | 34.23 | >200 | 26.87 | 455 (MH⁺, 100%) | 2.32 |
| 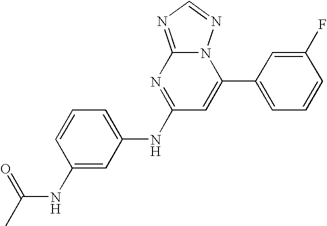 | 131 | 69.40 | >200 | 26.29 | 363 (MH⁺, 100%) | 2.14 |
| 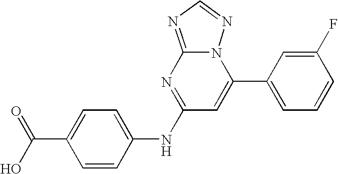 | 132 | 36.15 | >200 | >200 | 350 (MH⁺, 100%) | 2.17 |
| 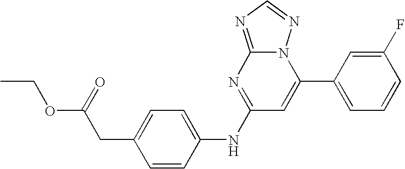 | 133 | 2.25 | >200 | 8.84 | 392 (MH⁺, 100%) | 2.45 |
| 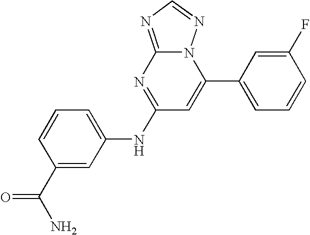 | 134 | 8.09 | >200 | 23.20 | 349 (MH⁺, 100%) | 2.02 |
| 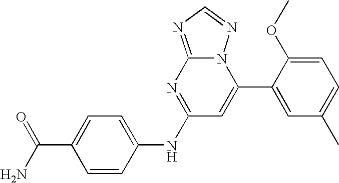 | 135 | 10.67 | >200 | 106.60 | 375 (MH⁺, 100%) | 2.00 |
| 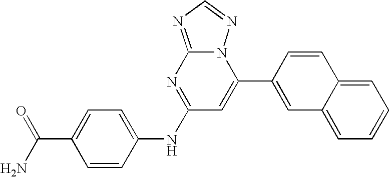 | 136 | 92.90 | >200 | 13.72 | 381 (MH⁺, 100%) | 2.19 |
| 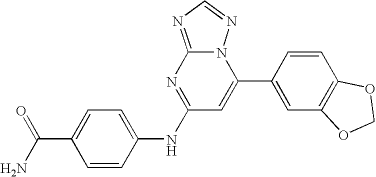 | 137 | 29.09 | >200 | 14.22 | 375 (MH⁺, 100%) | 1.94 |
| 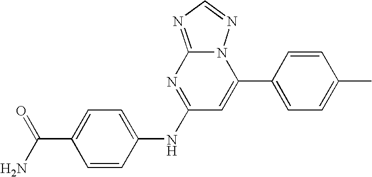 | 138 | 4.21 | >200 | 26.15 | 345 (MH⁺, 100%) | 2.02 |
| 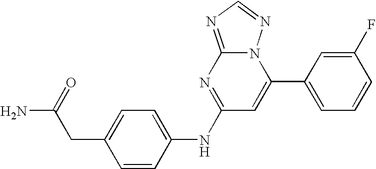 | 139 | 1.12 | >200 | 3.76 | 363 (MH⁺, 100%) | 1.99 |
| 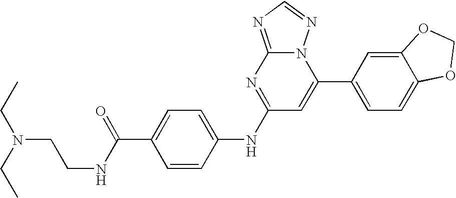 | 140 | 1.80 | >200 | 1.00 | 474 (MH⁺, 100%) | 1.79 |
| 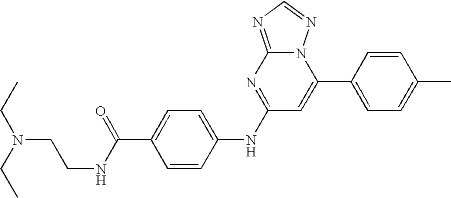 | 141 | 4.88 | >200 | 2.93 | 443 (MH⁺, 100%) | 1.88 |

TABLE 4

| | | | | | | RT |
|---|---|---|---|---|---|---|
| | | | | | | (min) |
| | Exam- | PDK1 | CDK2 | Chk1 | | (LC-MS |
| | ple | IC₅₀ | IC₅₀ | IC₅₀ | | Method |
| Structure | No. | (μM) | (μM) | (μM) | m/z | A) |
| 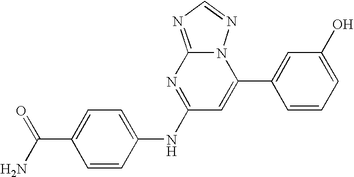 | 142 | 4.66 | | 3.92 | 347 (MH⁺, 100%) | 1.76 |
| 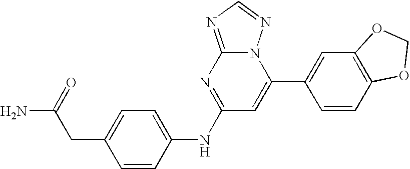 | 143 | 11.36 | | 4.71 | 389 (MH⁺, 100%) | 1.92 |
| 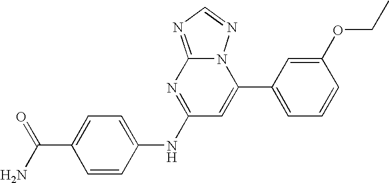 | 144 | 12.19 | | 68.28 | 375 (MH⁺, 100%) | 2.09 |
| 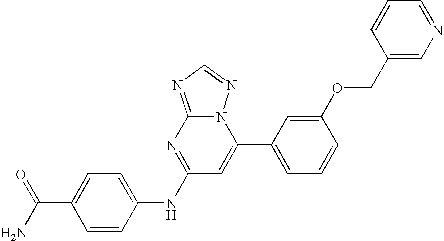 | 145 | 1.73 | 23.08 | 9.86 | 438 (MH⁺, 100%) | 1.88 |
| 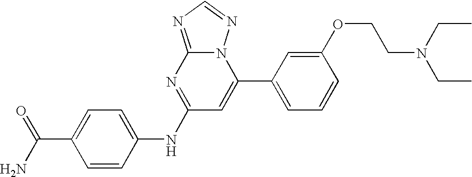 | 146 | 7.60 | 5.51 | 20.84 | 446 (MH⁺, 100%) | 1.56 |
| 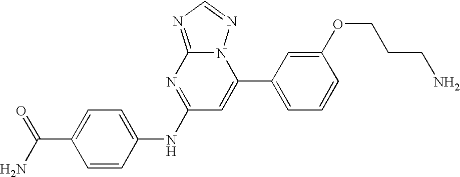 | 147 | 6.13 | 71.69 | 5.21 | 404 (MH⁺, 100%) | 1.52 |
| 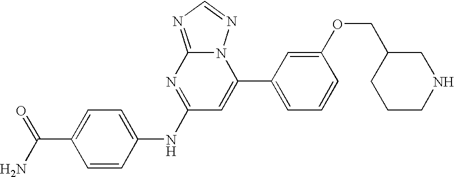 | 148 | 10.42 | 5.38 | 17.05 | 444 (MH⁺, 100%) | 1.59 |
| 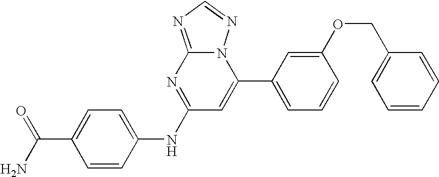 | 149 | 2.255 | 8.41 | 4.12 | 437 (MH⁺, 100%) | 2.33 |
| 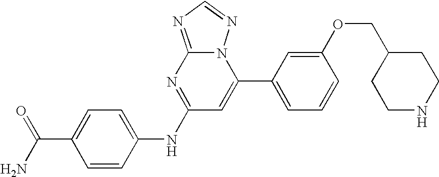 | 150 | 14.57 | | 19.74 | 444 (MH⁺, 100%) | 1.59 |
| 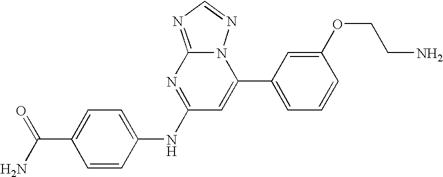 | 151 | 0.25 | 47.53 | 3.00 | 390 (MH⁺, 100%) | 1.48 |
| 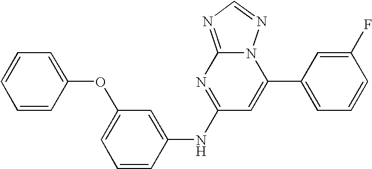 | 152 | 13.42 | | 32.74 | 398 (MH⁺, 100%) | 2.71 |
| 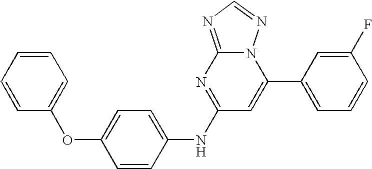 | 153 | 11.96 | | 24.73 | 398 (MH⁺, 100%) | 2.69 |
| 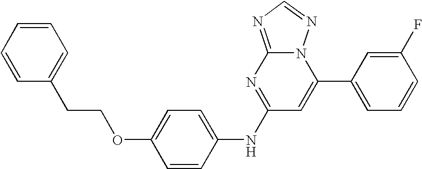 | 154 | 16.25 | | 27.32 | 412 (MH⁺, 100%) | 2.71 |
| 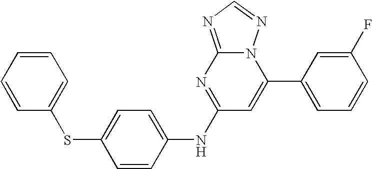 | 155 | 12.55 | | 15.08 | 414 (MH⁺, 100%) | 2.77 |
| 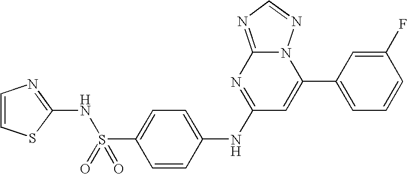 | 156 | 7.94 | | 13.02 | 468 (MH⁺, 100%) | 2.13 |
| 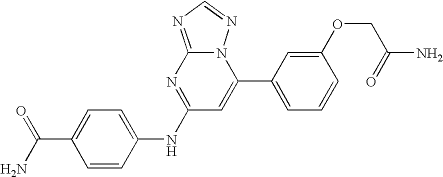 | 157 | 4.82 | 6.56 | 6.05 | 404 (MH⁺, 100%) | 1.68 |
| 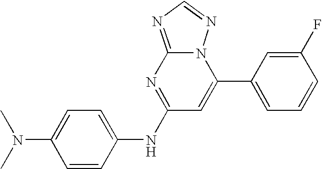 | 158 | 10.51 | | 6.75 | 349 (MH⁺, 100%) | 2.04 |
| 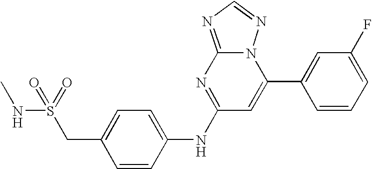 | 159 | 7.66 | | 22.62 | 413 (MH⁺, 100%) | 2.17 |
| 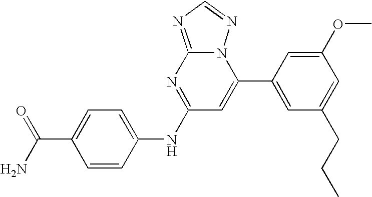 | 160 | 1.32 | | 15.84 | 403 (MH⁺, 100%) | 2.30 |
| 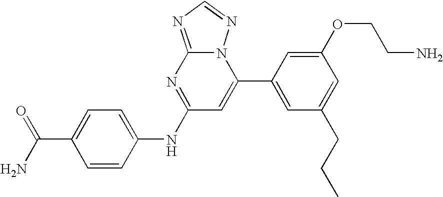 | 161 | 1.66 | | 9.04 | 432 (MH⁺, 100%) | 1.72 |
| 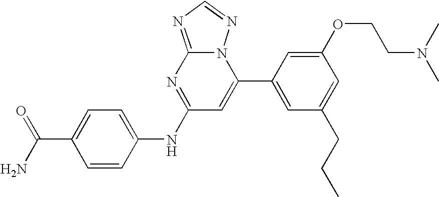 | 162 | 7.34 | | 31.69 | 460 (MH⁺, 100%) | 1.75 |
| 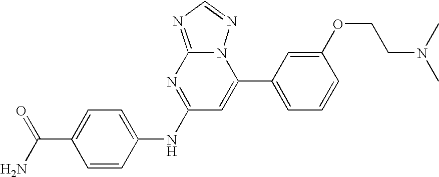 | 163 | 5.44 | | 9.82 | 418 (MH⁺, 100%) | 1.52 |
| 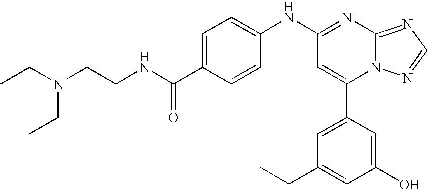 | 164 | 1.60 | | 0.66 | 474 (MH⁺, 100%) | 1.81 |
| 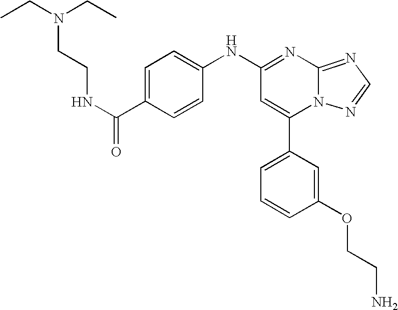 | 165 | 0.81 | 7.89 | 1.40 | 489 (MH⁺, 70%) and 245 [(M/2)H⁺], 100) | 1.45 |
| 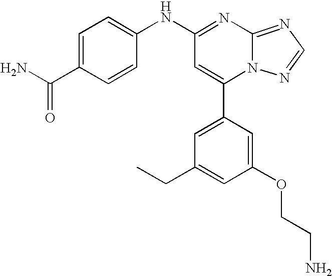 | 166 | 1.20 | 2.58 | 2.70 | 418 (MH⁺, 100%) | 1.64 |
| 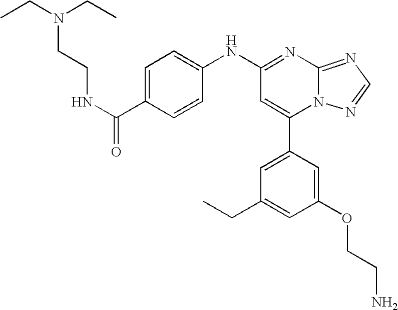 | 167 | 3.99 | | 3.39 | 517 (MH⁺, 70%) and 259 [(M/2)H⁺], 100) | 1.59 |

Assays

CDK2

Assays for the cyclin dependent kinase activity were carried out by monitoring the phosphorylation of a synthetic peptide, HATTPKKKRK. The assay mixture containing the inhibitor and CDK2 enzyme, complexed with cyclin A (0.4 U/ml) was mixed together in a microtiter plate in a final volume of 50 μl and incubated for 40 min at 30° C. The assay mixture contained 0.1 mM unlabeled ATP, 0.01 μCi/μl ³³P-γ-ATP, 0.03 mM peptide, 0.1 mg/ml BSA, 7.5 mM magnesium acetate, 50 mM HEPES-NaOH, pH 7.5. The reaction was stopped by adding 50 μl of 50 mM phosphoric acid. 90 μl of the mixture were transferred to a pre-wetted 96-well Multiscreen MAPHNOB filtration plate (Millipore) and filtered on a vacuum manifold. The filter plate was washed with three successive additions of 200 μl 50 mM phosphoric acid and then with 100 μl methanol. The filtration plate was dried for 10 min at 65° C., scintillant added and phosphorylated peptide quantified in a scintillation counter (Trilux, PerkinElmer).

HEPES is N-[2-hydroxyethyl]piperazine-N′-[2-ethanesulfonic acid]. BSA is bovine serum albumin.

PDK1

Assays for the 3′-phosphoinositide-dependent kinase activity were carried out by monitoring the phosphorylation of a synthetic peptide:KTFCGTPEYLAPEVRREPRILSEEEQEMFRDFDYIADWC.

The assay mixture containing the inhibitor and PDK1 enzyme was mixed together in a microtiter plate in a final volume of 50 μl and incubated for 60 min at 30° C. The assay mixture contained 0.01 mM unlabeled ATP, 0.01 μCi/μl ³³P-γ-ATP, 0.075 mM peptide, 0.1 mg/ml BSA, 7.5 mM magnesium acetate, 0.05 M Tris.HCl, pH 7.5, 0.5% 2-mercaptoethanol. The reaction was stopped by adding 50 μl of 50 mM phosphoric acid. 90 μl of the mixture were transferred to a pre-wetted 96-well Multiscreen MAPHNOB filtration plate (Millipore) and filtered on a vacuum manifold. The filter plate was washed with three successive additions of 200 μl 50 mM phosphoric acid and then with 100 μl methanol. The filtration plate was dried for 10 min at 65° C., scintillant added and phosphorylated peptide quantified in a scintillation counter (Trilux, PerkinElmer).

Chk1

Assays for the Chk1 kinase activity were carried out by monitoring the phosphorylation of a synthetic peptide Chktide with the amino acid sequence, KKKVSRSGLYRSPSMPENLNRPR. The assay mixture containing the inhibitor and Chk1 enzyme was mixed together in a microtiter plate in a final volume of 50 μl and incubated for 40 minutes at 30° C.

The assay mixture contained 0.01 mM unlabeled ATP, 0.5 μCi ³³P-γ-ATP, 30 μM Chktide, 0.1 mg/ml BSA, 50 mM Hepes-NaOH pH 7.5 and 11 nM GST-Chk1 enzyme. The reaction was stopped by adding 50 μl of 50 mM phosphoric acid. 90 μl of the mixture was transferred to a pre-wetted 96-well Multiscreen MAPHNOB filtration plate (Millipore) and filtered on a vacuum manifold. The filter plate was washed with three successive additions of 200 μl 50 mM phosphoric acid and then with 100 μl methanol. The filtration plate was dried for 10 min at 65° C., scintillant added and phosphorylated peptide quantified in a scintillation counter (Trilux, PerkinElmer).

1. A compound of formula (IA) or (IB) or a salt, N-oxide, hydrate or solvate thereof: 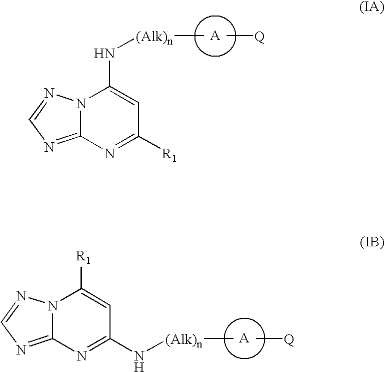
wherein
Ring A is an optionally substituted aryl, heteroaryl, carbocyclic, or heterocyclic radical,
Alk represents an optionally substituted divalent C1-C₆ alkylene radical;
n is 0 or 1;
Q represents a radical of formula -(Alk¹)ₚ-(X)ᵣ-(Alk²)ₛ-Z wherein in any compatible combination
Z is hydrogen or an optionally substituted carbocyclic or heterocyclic ring,
Alk¹ and Alk² are optionally substituted divalent C₁-C₆ alkylene radicals which may contain a —O—, —S—, or —NR^{A}— link, wherein R^{A} is hydrogen or C₁-C₆ alkyl,
X represents —O—, —S—, —(C═O)—, —(C═S)—, —SO₂—, —SO—, —C(═O)O—, —OC(═O)—, —C(═O)NRA-, —NR^{A}C(═O)—, —C(═S)NR^{A}—, —NR^{A}C(═S)—, —SO₂NR^{A}, —NR^{A}SO₂—, —OC(═O)NR^{A}, —NR^{A}C(═O)O—, or —NR^{A}— wherein R^{A} is hydrogen or C₁-C₆ alkyl,
p, r and s are independently 0 or 1, and
R₁ represents a radical -(Cyc)ₖ(Alk³)ₐ-(Y)_{b}-(Alk⁴)ₖB wherein
k, a, b, and d are independently 0 or 1,
Cyc represents monocyclic divalent carbocyclic or heterocyclic radical having from 5 to 8 ring atoms
Alk³ and Alk⁴ are optionally substituted divalent C₁-C₃ alkylene radicals,
Y represents a monocyclic divalent carbocyclic or heterocyclic radical having from 5 to 8 ring atoms, —O—, —S—, or —NR^{A} wherein R^{A} is hydrogen or Cₗ-C₆ alkyl,
B represents hydrogen or halo, or an optionally substituted monocyclic, carbocyclic or heterocyclic ring with 5 or 6 ring members, or in the case where Y is —NR^{A} and b is 1, then R^{A} and the radical -(Alk⁴)_{d}-B taken together with the nitrogen to which they are attached may form an optionally substituted heterocyclic ring.

2. The compound as claimed in claim 1 wherein n is 0

3. The compound as claimed in claim 1 wherein n is 1 and Alk is methyl or ethyl.

4. The compound as claimed in any of claim 1 wherein Q is hydrogen and ring A is optionally substituted phenyl, naphthyl, 2-,3- or 4-pyridyl, 5-pyrimidinyl, 2- or 3-thienyl, 2- or 3-furyl.

5. The compound as claimed in claim 1 wherein Q is hydrogen and ring A is optionally substituted phenyl.

6. The compound as claimed in claim 1 wherein Q is hydrogen and ring A is substituted by methyl, ethyl, methylenedioxy, ethylenedioxy, methoxy, ethoxy, methylthio, ethylthio, hydroxy, hydroxymethyl, hydroxyethyl, mercapto, mercaptomethyl, mercaptoethyl, amino, mono- or di-methylamino, mono- or di-ethylamino, fluoro, chloro, bromo, cyano, dimethylsulfonyl, N-morpholino, N-piperidinyl, or N-piperazinyl, the latter being optionally C₁-C₆ alkyl- or benzyl-substituted on the free ring nitrogen.

7. The compound as claimed in claim 1 wherein, in the Q substituent, p, r and s are each 0, and Z is an optionally substituted phenyl, cyclopentyl, cyclohexyl, pyridyl, morpholino, piperidinyl, or piperazyl ring.

8. The compound as claimed in claim 1 wherein, in the Q substituent, one or more of p, r, and s is/are 1, and Z is hydrogen or an optionally substituted carbocyclic or heterocyclic ring.

9. The compound as claimed in claim 8 wherein p, s, or both are 1 and r is 0.

10. The compound as claimed in claim 8 wherein p, r, and s are 1.

11. The compound as claimed in claim 8 wherein p and s are 0 and r is 1.

12. The compound as claimed in claim 11 wherein ring A is phenyl with Q in the 4-position, X is —SO₂—, and Z is optionally substituted phenyl.

13. The compound as claimed in claim 8 wherein p is 0 or 1, r is 1, and X is a sulfonyl —SO₂— radical, or a sulfonamide radical —NR^{A}SO₂—, or a carboxamide radical —NR^{A}C(═O)— wherein R^{A} is hydrogen or (C₁-C₆)alkyl, with the N atom linked to the ring A.

14. The compound as claimed in claim 13 wherein s is 1 and Z is hydrogen.

15. The compound as claimed in claim 13 wherein s is 0 and Z is an optionally substituted carbocyclic or heterocyclic ring.

16. The compound as claimed in claim 14 wherein Z is phenyl, optionally substituted in the 4-position.

17. The compound as claimed in any claim 1 wherein, in the substituent R₁, k, a, b, and d are all 0, and B is hydrogen or halo.

18. The compound as claimed in claim 1 wherein, in the substituent R₁, B is optionally substituted cyclopentyl, cyclohexyl, phenyl, 2-, 3-, or 4-pyridyl, 2-, or 3-thienyl, 2-, or 3-furanyl, pyrrolyl, pyranyl, or piperidinyl ring.

19. The compound as claimed in claim 18 wherein the optional substituents are selected from methyl, ethyl, methoxy, ethoxy, methylenedioxy, ethylenedioxy, methylthio, ethylthio, hydroxy, hydroxymethyl, hydroxyethyl, mercapto, mercaptomethyl, mercaptoethyl, amino, mono- and di-methylamino, monoand di-ethylamino, fluoro, chloro, bromo, cyano, N-morpholino, N-piperidinyl, N-piperazinyl, the latter being optionally C₁-C₆ alkyl- or benzyl-substituted on the free ring nitrogen.

20. The compound as claimed in claim 18 wherein B is 4-amino-cyclohexyl or 4amino-piperidin-1-yl.

21. The compound as claimed in claim 19 wherein b and k are 0 and a, c, or both are 1.

22. The compound as claimed in claim 19 wherein k, a and d are 0, b is 1, and Y —O—, —S—, or —NR^{A} wherein R^{A} is hydrogen or C₁-C₆ alkyl.

23. The compound as claimed in claim 18 wherein k and b are 0, at least one of a and d is 1, and B is hydrogen.

24. The compound as claimed in claim 18 wherein k is 0, a is 1 or 0, b is 1, Y is —NR^{A}-, and the radical-(Alk⁴)ₖ-B taken together with —R^{A} and the nitrogen to which they are attached form an optionally substituted piperidinyl, morpholinyl, or piperazinyl ring.

25. The compound as claimed in claim 18 wherein k is 1, Cyc is a phenylene radical, and Y, if present, is non-cyclic.

26. A method of treatment of diseases or conditions mediated by excessive or inappropriate kinase activity in mammals comprising administering to the mammal an amount of a compound of formula (IA) or (IB) as defined in claim 1, or a salt, hydrate or solvate thereof, effective to inhibit said kinase activity.

27. (canceled)

28. The method as claimed in claim 26, wherein the kinase activity is at least one selected from CDK2, PDK1, and Chk1 activity.

29. The method of treatment as claimed in claim 26, wherein the kinase activity is associated with cancer, psoriasis, or restenosis.

30. (canceled)

31. A pharmaceutical composition comprising a compound as claimed in claim 1, together with a pharmaceutically acceptable carrier.

32. The method of claim 26 wherein the mammals are humans.
